(19) United States
(12) Patent Application Publication
(10) Pub. No.: US 2024/0324644 A1
(43) Pub. Date: Oct. 3, 2024

(54) Mix of specific Bifidobacterium species and specific non-digestible oligosaccharides

(71) Applicant: N.V. Nutricia, Zoetermeer (NL)

(72) Inventors: Cornelus Johannes Petrus van Limpt, Utrecht (NL); Kaouther Ben Amor, Utrecht (NL); Jan Knol, Utrecht (NL); Ana Maria GIL RODRÍGUEZ, Utrecht (NL)

(73) Assignee: N.V. Nutricia, Zoetermeer (NL)

(21) Appl. No.: 18/741,853

(22) Filed: Jun. 13, 2024

Related U.S. Application Data
(63) Continuation of application No. PCT/EP2022/087618, filed on Dec. 22, 2022.

(30) Foreign Application Priority Data
Dec. 22, 2021 (EP) 21217064.1

(51) Int. Cl. A23L 33/135; A23L 33/00; A23L 33/125

(52) U.S. Cl. CPC A23L 33/135; A23L 33/125; A23L 33/40

(57) ABSTRACT

A consortium of specific probiotic bacteria comprising Bifidobacterium bifidum and Bifidobacterium breve with specific carbohydrate degrading properties, specific human milk oligosaccharide and preferably also beta-galacto-oligosaccharides with a higher degree of polymerization, is particularly effective for improving intestinal microbiota resilience.

### FIELD OF THE INVENTION

The invention is in the field of nutritional compositions with probiotics and prebiotics for children for preventing and/or treating intestinal microbial dysbiosis and improving microbiota resilience.

### BACKGROUND

A healthy intestinal microbiota development in early life is known to lead to a well-balanced intestinal microbiota and an improved intestinal microbiota resilience later in life, i.e. the ability to return swiftly to the well-balanced state after a disruptive event. This effect is thought to have short and long term health advantages. Examples of such health effects are a reduction in infections, diarrhea and allergic manifestations. A balanced and resilient intestinal microbiota is further believed to beneficially impact the functioning of the immune system, the brain and to improve metabolic health later in life.

In healthy term, vaginally born infants a well-balanced intestinal microbiota develops naturally, driven by the inoculation with beneficial bacteria derived from the mother during birth and subsequently fueled specifically by the human milk oligosaccharides present in the human milk. When the infant ages, the microbiota will develop and diversify upon weaning. During childhood the microbiota will further develop and become more like the more stable microbiota as present in a healthy adult.

Examples of factors that may negatively affect the establishment of or disturb the presence of a well-balanced intestinal microbiota are delivery mode (caesarean delivery), preterm birth and antibiotic usage by the infant or by the lactating mother.

Because of the instability of the intestinal microbiota early in life and during childhood compared to the intestinal microbiota in adulthood and the long term health effects that perturbation on the intestinal microbiota early in life may have, it is of utmost importance to prevent or to treat intestinal microbial dysbiosis during childhood. This is in particular so for subjects that are not fully breastfed and do not receive the full potential dose of human milk oligosaccharides and therefore are at risk of intestinal microbial dysbiosis.

WO 2005/039319 discloses the combination of a Bifidobacterium breve species and at least two different non-digestible oligosaccharides for improving the intestinal microbiota early in life.

WO2007/045502 discloses the use of at least three different Bifidobacterium species to improve the Bifidobacterium biodiversity in infants born via Caesarean section.

Egan et al, 2014, BMC Microbiology 2014, 14:282, show cross-feeding by Bifidobacterium breve UCC2003 during co-cultivation with Bifidobacterium bifidum PRL2010 in a mucin-based medium.

WO 2019/055718 discloses use of compositions to increase output of particular metabolites in the gut of a nursing infant with one or more bacterial strains selected for their growth on mammalian milk oligosaccharides, a source of mammalian milk oligosaccharides, and, optionally, nutritive components required for the growth of that infant.

WO 2016/149149 discloses a composition comprising at least two non-pathogenic microbes, wherein one of the at least two non-pathogenic microbes is from a first species capable of internalizing and/or metabolizing dietary glycans, and wherein one of the at least two non-pathogenic microbes is from a second species capable of consuming and metabolizing free sugar monomers.

Toscano et al, 2015, Arch Microbiol 65:1079-1086 disclose a mix of B. breve M-16V, B. infantis M-63 and B. longum BB356 for growth compatibility.

Still there is a need for specific mixes with further improved effects on the intestinal microbiota and improving the intestinal microbial resilience in particular early in life in subjects suffering from or at risk of intestinal microbial dysbiosis.

### SUMMARY OF THE INVENTION

An aim of the inventors was to develop a consortium of different bifidobacterial species specific for infants and different types of non-digestible oligosaccharides that interact in a synergistic and syntrophic way. Such a consortium is suitable to be applied early in life to prevent or treat disruptions of the intestinal microbiota in subjects at risk of exposed to such disruptions.

Several single stains and several mixes of bifidobacteria were tested in combination with several single non-digestible oligosaccharides and mixes of non-digestible oligosaccharides. It was found that a combination of specific human milk oligosaccharides (HMO), such as 2′-fucosyllactose and Bifidobacterium bifidum, which is able to hydrolyze the specific HMO extracellularly, interacted in a beneficial syntrophic way with a Bifidobacterium breve that cannot grow on the specific HMO. This combination led to synergistic fermentation and growth of the two different bifidobacterium strains in the mix, and also improved prebiotic and anti-pathogenic properties. This synergetic effect was further increased when also galacto-oligosaccharides (GOS) comprising structures with a degree of polymerization (DP) of 4 or more were present and the Bifidobacterium breve strain has extracellular beta1,4 endogalactanase activity. This mix was showing a further improved synergistic fermentation, growth, and prebiotic and anti-pathogenic properties. Another further improvement was found using a third bifidobacterium strain being a B. breve without beta1,4 galactanase activity.

Combinations that contained other bifidobacterial species not having these properties did not show a synergistic effect to the same extent or even showed an antagonistic effect. For instance, combinations of B. breve strains with B. longum subsp. infantis and 2′-fucosyllactose showed antagonistic growth when combined.

Using an experimental model with microbiota from an infant that had been treated with antibiotics, it was found this specific combination of Bifidobacterium species and specific HMO and preferably GOS maintained these syntrophic properties in an environment of a disturbed microbiota.

A comparative study of the effect of specific HMO and GOS, with or without the specific combination of Bifidobacterium species, on faeces of a pair of twins, one born vaginally and the other via caesarean section, also allowing the comparison of antibiotic treated vs non-antibiotic microbiota, confirmed the beneficial effect on intestinal microbial dysbiosis.

### DETAILED DESCRIPTION

The invention thus concerns a nutritional composition comprising a mix of Bifidobacterium species and at least one human milk oligosaccharide, wherein
a. the Bifidobacterium species comprise at least i) a Bifidobacterium bifidum strain able to express at least one extracellular enzyme selected from a fucosidase and a sialidase, ii) a Bifidobacterium breve strain able to metabolize a saccharide selected from L-fucose and sialic acid, and
b. the human milk oligosaccharide is at least one selected from the group consisting of 2′-fucosyllactose, 3-fucosyllactose, 3′-sialyllactose and 6′-sialyllactose, and wherein the nutritional composition is not mammalian milk.

### Bifidobacterium Mix

The present nutritional composition comprises at least two strains of bifidobacteria, of which one is a Bifidobacterium bifidum that is able to express at least one extracellular enzyme selected from a fucosidase and a sialidase and a Bifidobacterium breve able to metabolize a saccharide selected from L-fucose and sialic acid and preferably able to express extracellular beta1,4 endogalactanase. The present nutritional composition preferably contains at least 2·10³ colony forming units (cfu) bifidobacteria per gram dry weight of the nutritional composition, more preferably at least 2·10⁴ cfu, even more preferably at least 2·10⁵ cfu bifidobacteria per gram dry weight of the nutritional composition. The present nutritional composition preferably contains 2·10³ to 2·10¹³ colony forming units (cfu) bifidobacteria per gram dry weight of the nutritional composition, preferably 2·10⁴ to 2·10¹², more preferably 2·10⁵ to 2·10¹⁰, most preferably 2·10⁵ to 2·10⁹ cfu bifidobacteria per gram dry weight of the nutritional composition.

Preferably each Bifidobacterium strain of the mix according to the invention is able to hydrolyze and metabolize lactose by lactase or beta1,4-galactosidase. This enables to metabolize the lactose that is a result of the degradation of the human milk oligosaccharide and of beta-galacto-oligosaccharides (bGOS) that are optionally present.

Bifidobacterium bifidum

The nutritional composition comprises a strain of Bifidobacterium bifidum. This species is key to the mix and is able to express extracellularly enzymes that degrade human milk oligosaccharides. In particular they are able to express at least one of a fucosidase and a sialidase and this ability enables the hydrolysis of one or more of the human milk oligosaccharides (HMO) 2′-fucosyllactose (2′-FL), 3-fucosyllactose (3-FL), 3′-sialyllactose (3′-SL), and 6′-sialyllactose (6′-SL). Thus the present invention can also be defined as a nutritional composition comprising a mix of Bifidobacterium species and at least one human milk oligosaccharide, wherein
a. the Bifidobacterium species comprise at least i) a Bifidobacterium bifidum strain able to express at least one extracellular enzyme that hydrolyses at least one of 2′-fucosyllactose (2′-FL), 3-fucosyllactose (3-FL), 3′-sialyllactose (3′-SL), and 6′-sialyllactose (6′-SL), ii) a Bifidobacterium breve strain able to metabolize a saccharide selected from L-fucose and sialic acid, and
b. the human milk oligosaccharide is at least one selected from the group consisting of 2′-fucosyllactose, 3-fucosyllactose, 3′-sialyllactose and 6′-sialyllactose, and wherein the nutritional composition is not mammalian milk.

In one embodiment the Bifidobacterium bifidum in the present nutritional composition is able to grow on 2′-FL and hydrolyses it extracellularly to fucose and lactose. In one embodiment the Bifidobacterium bifidum in the present nutritional composition is able to grow on 3-FL and hydrolyses it extracellularly to fucose and lactose. In one embodiment the Bifidobacterium bifidum in the present nutritional composition is able to grow on 3′-SL and hydrolyses it extracellularly to sialic acid and lactose. In one embodiment the Bifidobacterium bifidum in the present nutritional composition is able to grow on 6′-SL and hydrolyses it extracellularly to sialic acid and lactose. Lactose is taken up by a transport system and split internally by a lactase into glucose and galactose. Bifidobacterium bifidum is not able to take up and metabolize fucose and sialic acid. These degradation products are therefore available for other bacteria to use as carbon and energy source.

Bifidobacterium bifidum is a Gram-positive, anaerobic, branched rod-shaped bacterium. The B. bifidum according to the present invention preferably has at least 95% identity of the 16 S rRNA sequence when compared to the type strain of B. bifidum ATCC 29521, more preferably at least 97% identity (Stackebrandt & Goebel, 1994, Int. J. Syst. Bacteriol. 44:846-849). Preferred B. bifidum strains are those isolated from the faeces of healthy human milk-fed infants. Typically, these are commercially available from producers of lactic acid bacteria, but they can also be directly isolated from faeces, identified, characterised and produced. Examples of suitable and available B. bifidum strains are B. bifidum R0071 from Lallemand or B. bifidum Bb-06 (Dupont Dansico). Most preferably, the B. bifidum is B. bifidum CNCM I-4319. This strain was deposited under Budapest treaty at the Collection National de Cultures de Microorganisms (CNCM) at Institut Pasteur, 25 Rue de Dr Roux, 75724 Paris by Compagnie Gervais Danone on 19 May 2010. B. bifidum CNCM I-4319 is a strain originally isolated from the infant microbiota of a healthy baby born in the Netherlands. This strain is especially preferred because it has the ability to protect the intestinal epithelial barrier measured by transepithelial electrical resistance (TEER) in an in vitro model (WO 2011/148358) and in an animal model it was shown to restore gut integrity and functionality from stress-induced and inflammatory damage (Tondereau at al., Microorganisms, 2020, 8, 1313). This is a characteristic that is especially beneficial under conditions when the intestinal microbiota is in disbalance. B. bifidum CNCM 1-4319 has also been disclosed in U.S. Pat. No. 9,402,872.

The present nutritional composition preferably contains at least 10³ cfu B. bifidum per gram dry weight of the nutritional composition, more preferably at least 10⁴ cfu, even more preferably 10⁵ cfu B. bifidum per gram dry weight of the nutritional composition. The present nutritional composition preferably contains 10³ to 10¹³ colony forming units (cfu) B. bifidum per gram dry weight of the nutritional composition, preferably 10⁴ to 10¹² cfu, more preferably 10⁵ to 10¹⁰ cfu, most preferably 10⁵ to 10⁹ cfu B. bifidum per gram dry weight of the nutritional composition.

Bifidobacterium breve

The nutritional composition comprises a strain of Bifidobacterium breve. In particular a B. breve that is able to metabolize a saccharide selected from L-fucose and sialic acid. Preferably the B. breve is also able to express extracellular beta1,4endogalactanase. This species is key to the mix as it is able to take up and metabolize fucose and/or sialic acid produced by the B. bifidum, but is not able to directly grow on 2′-FL, 3-FL, 3′-SL, or 6′-SL. Thus in a preferred embodiment according to the invention the Bifidobacterium breve strain or strains is/are not able to express fucosidase and is/are not able to express sialidase, or in other words is/are not able to express an enzyme that hydrolyses 2′-fucosyllactose (2′-FL), 3-fucosyllactose (3-FL), 3′-sialyllactose (3′-SL) and 6′-sialyllactose (6′-SL). The preferred ability to express extracellular beta1,4endogalactanase results in that the B. breve is able to release the galactose units from beta-galacto-oligosaccharides (bGOS) that have a degree of polymerization (DP) of 4 or above. A syntrophic effect was observed when B. bifidum and B. breve were combined. However, this effect was not observed, and even an antagonistic effect was observed when B. breve was combined with an other species of Bifidobacterium that is able to utilize human milk oligosaccharides such as 2′-FL, 3-FL, 3′-SL, or 6′-SL, namely Bifidobacterium longum subsp. infantis (B. infantis).

Bifidobacterium breve is a Gram-positive, anaerobic, branched rod-shaped bacterium. The B. breve according to the present invention preferably has at least 95% identity of the 16 S rRNA sequence when compared to the type strain of B. breve ATCC 15700, more preferably at least 97% identity (Stackebrandt & Goebel, 1994, Int. J. Syst. Bacteriol. 44:846-849).

Preferred B. breve strains are those isolated from the faeces of healthy human milk-fed infants. Typically these are commercially available from producers of lactic acid bacteria, but they can also be directly isolated from faeces, identified, characterised and produced. Suitable B. breve strains are available.

B. breve strains can be divided into two groups. A group that expresses extracellular beta1,4endogalactanase (encoded by the GalA gene) EC3.2.1.89, and strains that do not express this enzyme. B. breve strains that are able to express beta1,4endogalactanase are able to grow well on bGOS comprising oligosaccharides with a DP of 4 or more, because they are able to utilize all the bGOS components, whereas B. breve strains that are beta1,4endogalactanase negative grow to a lesser extent on bGOS as they are not able to degrade the bGOS with a high degree of polymerization. Beta1,4 endogalactanase is able to degrade galactan or potato derived pectic arabinogalactan into bGOS with a DP3. The galactotriose is subsequently transported into the cell and metabolized.

Whether a B. breve strain has the ability to express beta1,4endogalactanase can be determined by a growth experiment on bGOS and analysis of the supernatant as described in O'Connel Motherway et al 2012 Microbial biotechnology, 6: 67-69.

Examples of suitable B. breve strains that express beta1,4endogalactanase are B. breve UCC2003 (NCIMB 8807), C50, JCM7017, NCFB2258 and NCIMB8815. [JCM: Japan Collection of Microorganisms. LMG: BCCM/LMG Belgian Coordinated Collections of Microorganisms. NCFB, National collection of food bacteria, UK. NCIMB, National Collection of Industrial, Food and marine bacteria, UK]

Especially preferred is to use B. breve C50. B. breve C50 was deposited under deposit number CNCM I-2219, under the Budapest Treaty at the Collection Nationale de Cultures de Microorganism, at Institut Pasteur, 25 Rue du Dr Roux, Paris, France on 31 May 1999 by Compagnie Gervais Danone. This strain was published in WO 2001/001785 and in U.S. Pat. No. 7,410,653. This strain is known to have immune stimulating activity and is able to improve the microbiota by reducing pathogens like Clostridium, in particular Clostridium perfringens, and Bacteroides fragilis. Furthermore the strain can produce factors that downregulate intestinal inflammation (Heuvelin et al 2009, Plos one, 4: e5184). These are features that are especially beneficial under conditions when the intestinal microbiota is in disbalance. Another preferred Bifidobacterium breve to use is Bifidobacterium breve CNCM 1-5177. B. breve CNCM 1-5177 was deposited under the Budapest Treaty at the Collection Nationale de Cultures de Microorganism, at Institut Pasteur, 25 Rue du Dr Roux, Paris, France on 9 Mar. 2017 by Compagnie Gervais Danone.

Although the impact of having a B. breve that is beta1,4endogalactanase positive is most prominent in the presence of galactans or bGOS comprising molecules with a DP of 4 or more, surprisingly also merely in the presence of human milk oligosaccharides such as 2′-FL as sole carbon and energy source the combination of a beta1,4endogalactanase positive strain like B. breve C50 and B. bifidum resulted in a higher acid formation than when a B. breve strain without beta1,4endogalactanase was used.

Preferably the nutritional composition according to the invention contains a combination of a strain of B. breve that is able to express extracellular beta1,4endogalactanase and a strain that is not able to express beta1,4endogalactanase. It was found that the combination of a B. bifidum, a B. breve able to express an active beta1,4endogalactanase and a B. breve not able to express beta1,4endogalactanase showed unexpectedly a further improved effect on intestinal microbial health, even under conditions where there was no bGOS comprising molecules with DP4 or higher present. The B. breve strains not able to express beta1.4 endogalactanase are able to grow on the degradation products like fucose, sialic acid and lactose and the shorter chain bGOS.

Suitable strains of B. breve that do not express beta1,4endogalactanase are JCM7019, LMG13208, NCFB2257, NCIMB11815, ATCC 15700, M-16V (LMG 23729, Morinaga).

Especially preferred is the M-16V strain. This strain, in the presence of non-digestible oligosaccharides including bGOS has demonstrated to increase the Bifidobacterium species diversity in infants that are C-section born and consequently have a microbial dysbiosis (Chua et al, 2017, JPGN 65: 102-106).

The present nutritional composition preferably contains at least 10³ cfu B. breve per gram dry weight of the nutritional composition, more preferably at least 10⁴ cfu, even more preferably at least 10⁵ cfu B. breve per gram dry weight of the nutritional composition. The present nutritional composition preferably contains 10³ to 10¹³ colony forming units (cfu) B. breve per gram dry weight of the nutritional composition, preferably 10⁴ to 10¹² cfu, more preferably 10⁵ to 10¹⁰ cfu most preferably from 10⁵ to 10⁹ cfu B. breve per gram dry weight of the nutritional composition.

The present nutritional composition preferably contains at least 10³ cfu B. breve able to express beta1,4endogalactanase per gram dry weight of the nutritional composition, more preferably at least 10⁴ cfu, even more preferably at least 10⁵ cfu B. breve able to express beta1,4endogalactanase per gram dry weight of the nutritional composition. The present nutritional composition preferably contains 10³ to 10¹³ colony forming units (cfu) B. breve able to express beta1,4endogalactanase per gram dry weight of the nutritional composition, preferably 10⁴ to 10¹² cfu, more preferably 10⁵ to 10¹⁰ cfu most preferably from 10⁵ to 10⁹ cfu B. breve able to express beta1,4endogalactanase per gram dry weight of the nutritional composition.

In one embodiment, the present nutritional composition preferably contains at least 10³ cfu B. breve not able to express beta1,4endogalactanase per gram dry weight of the nutritional composition, more preferably at least 10⁴ cfu, even more preferably at least 10⁵ cfu B. breve not able to express beta1,4endogalactanase per gram dry weight of the nutritional composition. The present nutritional composition preferably contains 10³ to 10¹³ colony forming units (cfu) B. breve not able to express beta1,4endogalactanase per gram dry weight of the nutritional composition, preferably 10⁴ to 10¹² cfu, more preferably 10⁵ to 10¹⁰ cfu most preferably from 10⁵ to 10⁹ cfu B. breve not able to express beta1,4endogalactanase per gram dry weight of the nutritional composition.

The present nutritional composition preferably contains both at least 10³ cfu B. breve able to express beta1,4endogalactanase per gram dry weight of the nutritional composition and at least 10³ cfu B. breve not able to express beta1,4endogalactanase per gram dry weight of the nutritional composition, more preferably at least 10⁴ cfu of each of both B. breve strains, even more preferably at least 10⁵ cfu of each of both B. breve strains per gram dry weight of the nutritional composition. The present nutritional composition preferably contains 10³ to 10¹³ colony forming units (cfu) of B. breve able to express beta1,4endogalactanase and 10³ to 10¹³ colony forming units (cfu) of B. breve not able to express beta1,4endogalactanase per gram dry weight of the nutritional composition, preferably 10⁴ to 10¹² cfu, more preferably 10⁵ to 10¹⁰ cfu, most preferably from 10⁵ to 10⁹ cfu of each of both B. breve strains per gram dry weight of the nutritional composition.

In a preferred embodiment, the Bifidobacterium breve strain, preferably the B. breve able to express beta1,4endogalactanase, is able to metabolize L-fucose. In a further preferred embodiment, if present, also the B. breve not able to express beta1,4endogalactanase is able to metabolize L-fucose.

For enablement of the present invention hereabove various bifidobacterial strains have been specifically identified. It is to be understood that the present invention is also enabled for bifidobacteria strains that are not identical to those specifically mentioned, but that have the same functional ability. In particular a natural variant or mutant of the specific Bifidobacterium bifidum mentioned above and that is capable of expressing at least one extracellular enzyme selected from a fucosidase and a sialidase, or in other words capable of expressing fucosidase and/or sialidase activity, is also encompassed by the present invention. Likewise, a natural variant or mutant of the specific Bifidobacterium breve mentioned above and that is capable to metabolize a saccharide selected from L-fucose and sialic acid is also encompassed by the present invention. Moreover, a natural variant or mutant of the specific Bifidobacterium breve mentioned above and that is capable to metabolize a saccharide selected from L-fucose and sialic acid and that is also capable of expressing extracellular beta1,4endogalactanase, or in other words capable of expressing beta1,4endogalactanase activity, is also encompassed by the present invention.

In the context of the present invention it is noted that ability to express extracellular fucosidase, sialidase or beta1,4endogalactanase means that such enzyme activity is expressed extracellularly.

Bifidobacterium longum subsp. infantis

Optionally the nutritional composition comprises additionally a strain of Bifidobacterium longum subsp. infantis (B. infantis). Although a combination of B. infantis with B. breve alone shows an antagonistic effect on growth and fermentation when grown on the HMO, in the presence of B. bifidum the antagonism has disappeared and there appears to be no negative effect on the synergistic interaction between B. bifidum and B. breve. This allows to optionally include a selected B. infantis strain that has known additional health effects.

Bifidobacterium longum subsp. infantis is a Gram-positive, anaerobic, branched rod-shaped bacterium. The present B. infantis preferably has at least 95% identity of the 16 S rRNA sequence when compared to the type strain of B. longum subsp. infantis ATCC 15607, more preferably at least 97% identity (Stackebrandt & Goebel, 1994, Int. J. Syst. Bacteriol. 44:846-849). In one embodiment, the nutritional composition according to the invention further comprises a strain of Bifidobacterium longum subspecies infantis. Preferably the strain of Bifidobacterium longum subspecies infantis is able to internalize a human milk oligosaccharides selected from the group consisting of 2′-fucosyllactose, 3-fucosyllactose, 3′-sialyllactose, and 6′-sialyllactose.

Preferred B. infantis strains to be optionally included in the nutritional composition according to the present invention are those isolated from the faeces of healthy human milk-fed infants. Typically, these are commercially available from producers of lactic acid bacteria, but they can also be directly isolated from faeces, identified, characterised and produced. B. infantis strains are available.

Suitable and available B. infantis strains are B. infantis M63 (LMG 23728, Morinaga), BB-02 (Christian Hansen, DSM 33361), or R0033 (Lallemand).

If present, the present nutritional composition preferably contains at least 10³ cfu B. infantis per gram dry weight of the nutritional composition, more preferably at least 10⁴ cfu even more preferably at least 10⁵ cfu B. infantis per gram dry weight of the nutritional composition. If present, the present nutritional composition preferably contains 10³ to 10¹³ colony forming units (cfu) B. infantis per gram dry weight of the present composition, preferably 10⁴ to 10¹² cfu more preferably 10⁵ to 10¹⁰ cfu most preferably from 10⁵ to 10⁹ cfu B. infantis per gram dry weight of the nutritional composition.

In one embodiment of the present invention the nutritional composition does not comprise a B. longum subsp. infantis since the synergistic and syntrophic effects were already present in the absence of B. infantis strain.

Preferably the nutritional composition of the present invention does not comprise B. longum subs. longum (B. longum). There was no contribution to the syntrophic effect found. As the syntrophic effect is very specific for the mix of B. bifidum and B. breve, preferably a beta1,4endogalactanase positive B. breve, more preferably a combination of a beta1,4endogalactanase positive and negative B. breve, and optionally further comprising B. infantis, addition of further species may disturb the syntrophic interaction.

Preferably the nutritional composition of the present invention does not contain lactobacilli, or in other words, does not comprise a Lactobacillus species. As the syntrophic effect is very specific for the mix of B. bifidum and B. breve, preferably a beta1,4endogalactanase positive B. breve, more preferably a combination of a beta1,4endogalactanase positive and negative B. breve, and optionally further comprising B. infantis, addition of further species may disturb the syntrophic interaction. Furthermore, lactobacilli are a relatively minor component early in life of a balanced microbiota compared to bifidobacteria.

The present nutritional composition preferably contains at least 10³ cfu B. bifidum and at least 10³ cfu B. breve, preferably B. breve able to express beta1,4endogalactanase, per gram dry weight of the nutritional composition, more preferably at least 10⁴ cfu of each of B. bifidum and B. breve, preferably B. breve able to express beta1,4endogalactanase, even more preferably 10⁵ cfu of each of B. bifidum and B. breve, preferably B. breve able to express beta1,4endogalactanase, per gram dry weight of the nutritional composition. The present nutritional composition preferably contains 10³ to 10¹³ colony forming units (cfu) B. bifidum and 10³ to 10¹³ colony forming units (cfu) B. breve, preferably B. breve able to express beta1,4endogalactanase, per gram dry weight of the nutritional composition, preferably 10⁴ to 10¹² cfu of each of B. bifidum and B. breve, preferably B. breve able to express beta1,4endogalactanase, more preferably 10⁵ to 10¹⁰ cfu, most preferably 10⁵ to 10⁹ cfu of each of B. bifidum and B. breve, preferably B. breve able to express beta1,4endogalactanase, per gram dry weight of the nutritional composition.

In one embodiment, the present nutritional composition preferably contains at least 10³ cfu B. bifidum and at least 10³ cfu B. breve able to express beta1,4endogalactanase and at least 10³ cfu B. breve not able to express beta1,4endogalactanase per gram dry weight of the nutritional composition, more preferably at least 10⁴ cfu of each of B. bifidum and B. breve able to express beta1,4endogalactanase and B. breve not able to express beta1,4endogalactanase, even more preferably 10⁵ cfu of each of B. bifidum and B. breve able to express beta1,4endogalactanase and B. breve not able to express beta1,4endogalactanase per gram dry weight of the nutritional composition.

The present nutritional composition preferably contains 10³ to 10¹³ colony forming units (cfu) B. bifidum and 10³ to 10¹³ colony forming units (cfu) B. breve able to express beta1,4endogalactanase and 10³ to 10¹³ colony forming units (cfu) B. breve not able to express beta1,4endogalactanase per gram dry weight of the nutritional composition, preferably 10⁴ to 10¹² cfu of each of B. bifidum and B. breve able to express beta1,4endogalactanase and B. breve not able to express beta1,4endogalactanase, more preferably 10⁵ to 10¹⁰ cfu, most preferably 10⁵ to 10⁹ cfu of each of B. bifidum and B. breve able to express beta1,4endogalactanase and B. breve not able to express beta1,4endogalactanase per gram dry weight of the nutritional composition.

In one embodiment, the present nutritional composition preferably contains at least 10³ cfu B. bifidum and at least 10³ cfu B. breve, preferably B. breve able to express beta1,4endogalactanase, and at least 10³ cfu B. infantis per gram dry weight of the nutritional composition, more preferably at least 10⁴ cfu of each of B. bifidum and B. breve, preferably B. breve able to express beta1,4endogalactanase, and B. infantis, even more preferably 10⁵ cfu of each of B. bifidum and B. breve, preferably B. breve able to express beta1,4endogalactanase, and B. infantis per gram dry weight of the nutritional composition.

The present nutritional composition preferably contains 10³ to 10¹³ colony forming units (cfu) B. bifidum and 10³ to 10¹³ colony forming units (cfu) B. breve, preferably B. breve able to express beta1,4endogalactanase, and 10³ to 10¹³ colony forming units (cfu) B. infantis per gram dry weight of the nutritional composition, preferably 10⁴ to 10¹² cfu of each of B. bifidum and B. breve, preferably B. breve able to express beta1,4endogalactanase, and B. infantis, more preferably 10⁵ to 10¹⁰ cfu, most preferably 10⁵ to 10⁹ cfu of each of B. bifidum and B. breve, preferably B. breve able to express beta1,4endogalactanase, and B. infantis per gram dry weight of the nutritional composition.

In one embodiment, the present nutritional composition preferably contains at least 10³ cfu B. bifidum and at least 10³ cfu B. breve able to express beta1,4endogalactanase and at least 10³ cfu B. breve not able to express beta1,4endogalactanase and at least 10³ cfu B. infantis per gram dry weight of the nutritional composition, more preferably at least 10⁴ cfu of each of B. bifidum and B. breve able to express beta1,4endogalactanase and B. breve not able to express beta1,4endogalactanase and B. infantis, even more preferably 10⁵ cfu of each of B. bifidum and B. breve able to express beta1,4endogalactanase and B. breve not able to express beta1,4endogalactanase and B. infantis per gram dry weight of the nutritional composition. The present nutritional composition preferably contains 10³ to 10¹³ colony forming units (cfu) B. bifidum and 10³ to 10¹³ colony forming units (cfu) B. breve able to express beta1,4endogalactanase and 10³ to 10¹³ colony forming units (cfu) B. breve not able to express beta1,4endogalactanase and 10³ to 10¹³ colony forming units (cfu) B. infantis per gram dry weight of the nutritional composition, preferably 10⁴ to 10¹² cfu of each of B. bifidum and B. breve able to express beta1,4endogalactanase and B. breve not able to express beta1,4endogalactanase and B. infantis, more preferably 10⁵ to 10¹⁰ cfu, most preferably 10⁵ to 10⁹ cfu of each of B. bifidum and B. breve able to express beta1,4endogalactanase and B. breve not able to express beta1,4endogalactanase and B. infantis per gram dry weight of the nutritional composition.

In a preferred embodiment, the nutritional composition according to the invention contains at least 10⁵ cfu B. bifidum per gram dry weight of the nutritional composition and at least 10⁵ cfu B. breve, preferably B. breve able to express beta1,4endogalactanase, per gram dry weight of the nutritional composition and optionally at least 10⁵ cfu B. breve not able to express beta1,4endogalactanase per gram dry weight of the nutritional composition, and optionally contains B. infantis, and wherein the total amount of bifidobacterium is at least 10⁶ cfu per gram dry weight of the nutritional composition.

In a preferred embodiment, the nutritional composition according to the invention contains at least 10⁵ cfu B. bifidum per gram dry weight of the nutritional composition and at least 10⁵ cfu B. breve able to express beta1,4endogalactanase per gram dry weight of the nutritional composition and at least 10⁵ cfu B. breve not able to express beta1,4endogalactanase per gram dry weight of the nutritional composition, and optionally contains B. infantis, and wherein the total amount of bifidobacteria is at least 10⁶ cfu per gram dry weight of the nutritional composition.

Preferably the nutritional composition contains B. bifidum and B. breve, preferably B. breve able to express extracellular beta1,4endogalactanase in a cfu ratio of 1:10³ to 10³:1, more preferably in a cfu ratio of 1:10² to 10²:1.

Preferably the nutritional composition contains B. bifidum, B. breve able to express extracellular beta1,4endogalactanase and B. breve not able to express beta1,4endogalactanase in a cfu ratio of (1-10³):(1-10³):(1:10³), more preferably (1-10²):(1-10²):(1:10²), meaning that each strain may differ by a factor 10³, preferably a factor 10², in cfu from any other strain present.

Preferably the nutritional composition contains B. bifidum, B. breve preferably able to express beta1,4endogalactanase and B. infantis in a cfu ratio of (1-10³):(1-10³):(1:10³)), more preferably (1-10²):(1-10²):(1:10²), meaning that each strain may differ by a factor 10³, preferably a factor 10², in cfu from any other strain present.

Preferably the nutritional composition contains B. bifidum, B. breve able to express beta1,4endogalactanase, B. breve not able to express beta1,4endogalactanase and B. infantis in a cfu ratio of (1-10³):(1-10³):(1:10³):(1:10³), more preferably (1-10²):(1-10²):(1:10²):(1:10²), meaning that each strain may differ by a factor 10³, preferably a factor 10², in cfu from any other strain present.

As mentioned before, preferably each Bifidobacterium strain of the mix according to the invention is able to hydrolyze and metabolize lactose by lactase or beta1,4-galactosidase. This enables to metabolize the lactose that is a result of the degradation of the human milk oligosaccharide and of beta-galacto-oligosaccharides (bGOS) that are optionally present. Hence this concerns the B. bifidum, the B. breve, able to express beta1,4endogalactanase, the B. breve not able to express beta1,4endogalactanase and the B. infantis.

### Non Digestible Oligosaccharides

The present nutritional composition comprises non-digestible oligosaccharides (NDO). The term “oligosaccharides” as used herein refers to saccharides with a degree of polymerization (DP) of 2 to 250, preferably a DP 2 to 100, more preferably 2 to 60, even more preferably 2 to 10. If oligosaccharide with a DP of 2 to 100 is included in the present nutritional composition, this results in compositions that may contain oligosaccharides with a DP of 2 to 5, a DP of 50 to 70 and/or a DP of 7 to 60. The term “non-digestible oligosaccharides” (NDO) as used in the present invention refers to oligosaccharides which are not digested in the intestine by the action of acids or digestive enzymes present in the human upper digestive tract, e.g. small intestine and stomach, but which are preferably fermented by the human intestinal microbiota. For example, glucose, galactose, sucrose, lactose, maltose and maltodextrins are considered digestible. Preferably the present non-digestible oligosaccharides are soluble. The term “soluble” as used herein, when having reference to polysaccharides, fibres or oligosaccharides, means that the substance is at least soluble according to the method described by L. Prosky et al., J. Assoc. Off. Anal. Chem. 71, 1017-1023 (1988).

### Human Milk Oligosaccharide

The nutritional composition of the present invention comprises at least one human milk oligosaccharide selected from the group consisting of 2′-FL, 3-FL, 3′-SL and 6′-SL. Human milk oligosaccharide are present in human milk and are non-digestible oligosaccharides. Preferably the nutritional composition according to the invention comprises at least 0.005 g/100 ml of the human milk oligosaccharide.

Preferably, a nutritional composition according to the invention comprises 0.005 g to 1.5 g human milk oligosaccharides (HMOs) per 100 ml, preferably 0.01 g to 1.5 g HMOs per 100 ml, more preferably 0.02 g to 0.75 g, even more preferably 0.04 g to 0.3 g HMOs per 100 ml. Based on dry weight, the present nutritional composition preferably comprises 0.038 wt. % to 12 wt. % HMOs, preferably 0.075 wt. % to 12 wt. % HMOs, more preferably 0.15 wt. % to 6 wt. % HMOs, even more preferably 0.3 wt. % to 2.5 wt. % HMOs. Based on energy, the present nutritional composition preferably comprises 0.008 to 2.5 g HMOs per 100 kcal, preferably 0.015 to 2.5 g HMOs per 100 kcal, more preferably 0.03 to 1.0 g HMOs per 100 kcal, even more preferably 0.06 to 0.5 g HMOs per 100 kcal. A lower amount of HMO will be less effective in stimulating growth of the bifidobacteria, whereas a too high amount will result in an increase the risk of osmotic diarrhea. This will counteract the beneficial effects of the mix on the intestinal health.

Preferably, a nutritional composition according to the invention comprises at least 0.005 g of the sum of 2′-FL, 3-FL, 3′-SL and 6′-SL per 100 ml, more preferably at least 0.01 g, more preferably at least 0.02 g, even more preferably at least 0.04 g of the sum of 2′-FL, 3-FL, 3′-SL and 6′-SL per 100 ml. Based on dry weight, the present nutritional composition preferably comprises at least 0.038 wt. % of the sum of 2′-FL, 3-FL, 3′-SL and 6′-SL, more preferably at least 0.075 wt. %, more preferably at least 0.15 wt. % of the sum of 2′-FL, 3-FL, 3′-SL and 6′-SL, even more preferably at least 0.3 wt. %. Based on energy, the present nutritional composition preferably comprises at least 0.008 g of the sum of 2′-FL, 3-FL, 3′-SL and 6′-SL per 100 kcal, more preferably at least 0.015 g per 100 kcal, more preferably at least 0.03 g per 100 kcal, even more preferably at least 0.06 per 100 kcal.

Preferably, a nutritional composition according to the invention comprises 0.01 g to 1.5 g of the sum of 2′-FL, 3-FL, 3′-SL and 6′-SL per 100 ml, more preferably 0.02 g to 0.75 g, even more preferably 0.04 g to 0.3 g of the sum of 2′-FL, 3-FL, 3′-SL and 6′-SL per 100 ml. Based on dry weight, the present nutritional composition preferably comprises 0.075 wt. % to 12 wt. % of the sum of 2′-FL, 3-FL, 3′-SL and 6′-SL, more preferably 0.15 wt. % to 6 wt. % of the sum of 2′-FL, 3-FL, 3′-SL and 6′-SL, even more preferably 0.3 wt. % to 2.5 wt. %. Based on energy, the present nutritional composition preferably comprises 0.015 to 2.5 g of the sum of 2′-FL, 3-FL, 3′-SL and 6′-SL per 100 kcal, more preferably 0.03 to 1.0 g per 100 kcal, even more preferably 0.06 to 0.5 g per 100 kcal.

2′-Fucosyllactose (2′-FL) is an oligosaccharide present in human milk (HMO). It is not present in bovine milk. It consists of three monose units, fucose, galactose and glucose linked together. Lactose is a galactose unit linked to a glucose unit via a beta1,4linkage. A fucose unit is linked to a galactose unit of a lactose molecule via an alpha1,2 linkage. 2′-FL is commercially available, for instance from Sigma-Aldrich or Christian Hansen-Jennewein. 2′-FL is split by extracellular enzymes of B. bifidum into lactose and fucose.

Preferably the nutritional composition according to the invention comprises 2′-FL. Preferably the nutritional composition according to the invention comprises as a HMOS essentially 2′-FL, that means at least 95 wt % of the HMOS consists of 2′-FL. Preferably, a nutritional composition according to the invention comprises at least 0.005 g 2′-FL per 100 ml, more preferably at least 0.01 g, more preferably at least 0.02 g, even more preferably at least 0.04 g 2′-FL per 100 ml. Based on dry weight, the present nutritional composition preferably comprises at least 0.038 wt. % 2′-FL, more preferably at least 0.075 wt. %, more preferably at least 0.15 wt. %, even more preferably at least 0.3 wt. % 2′-FL. Based on energy, the present nutritional composition preferably comprises at least 0.008 g 2′-FL per 100 kcal, more preferably at least 0.015 g, more preferably at least 0.03 g 2′-FL per 100 kcal, even more preferably at least 0.09 g 2′-FL per 100 kcal.

Preferably, a nutritional composition according to the invention comprises 0.01 g to 1 g 2′-FL per 100 ml, more preferably 0.02 g to 0.5 g, even more preferably 0.04 g to 0.2 g 2′-FL per 100 ml. Based on dry weight, the present nutritional composition preferably comprises 0.075 wt. % to 8 wt. % 2′-FL, more preferably 0.15 wt. % to 4 wt. % 2′-FL, even more preferably 0.3 wt. % to 1.5 wt. % 2′-FL. Based on energy, the present nutritional composition preferably comprises 0.015 to 1.5 g 2′-FL per 100 kcal, more preferably 0.03 to 0.75 g 2′-FL per 100 kcal, even more preferably 0.06 to 0.4 g 2′-FL per 100 kcal. A lower amount of 2′-FL will be less effective in stimulating growth of the bifidobacteria, whereas a too high amount may increase the risk of osmotic diarrhea. This will counteract the beneficial effects of the mix on the intestinal health.

In a preferred embodiment, the nutritional composition according to the invention comprises the human milk oligosaccharide 2′-fucosyllactose and the Bifidobacterium breve strain, preferably the B. breve able to express beta1,4endogalactanase, that is able to metabolize L-fucose. In a further preferred embodiment, if present, also the B. breve not able to express beta1,4endogalactanase is able to metabolize L-fucose.

3-Fucosyllactose (3-FL) is an oligosaccharide present in human milk (HMO). It is not present in bovine milk. It consists of three monose units, fucose, galactose and glucose linked together. Lactose is a galactose unit linked to a glucose unit via a beta1,4linkage. A fucose unit is linked to a galactose unit of a lactose molecule via or via an alpha-1,3 linkage to the glucose unit of a lactose. 3-FL is commercially available, for instance from Sigma-Aldrich or Christian Hansen-Jennewein. 3-FL is split by extracellular enzymes of B. bifidum into lactose and fucose.

Preferably, a nutritional composition according to the invention comprises at least 0.005 g 3-FL per 100 ml, more preferably at least 0.01 g, even more preferably at least 0.02 g 3-FL per 100 ml. Based on dry weight, the present nutritional composition preferably comprises at least 0.04 wt. % 3-FL, more preferably at least 0.075 wt. % 3-FL, even more preferably at least 0.15 wt. % 3-FL. Based on energy, the present nutritional composition preferably comprises at least 0.008 g 3-FL per 100 kcal, more preferably at least 0.015 g 3-FL per 100 kcal, even more preferably at least 0.03 g 3-FL per 100 kcal.

Preferably, a nutritional composition according to the invention comprises 0.005 g to 0.5 g 3-FL per 100 ml, more preferably 0.01 g to 0.25 g, even more preferably 0.02 g to 0.10 g 3-FL per 100 ml. Based on dry weight, the present nutritional composition preferably comprises 0.04 wt. % to 4 wt. % 3-FL, more preferably 0.075 wt. % to 2.0 wt. % 3-FL, even more preferably 0.15 wt. % to 0.75 wt. % 3-FL. Based on energy, the present nutritional composition preferably comprises 0.008 to 0.75 g 3-FL per 100 kcal, more preferably 0.015 to 0.04 g 3-FL per 100 kcal, even more preferably 0.03 to 0.2 g 3-FL per 100 kcal. A lower amount of 3-FL will be less effective in stimulating growth of the bifidobacteria, whereas a too high amount may increase the risk of osmotic diarrhea. This will counteract the beneficial effects of the mix on the intestinal health.

3′-Sialyllactose (3′-SL) is split by extracellular enzymes of B. bifidum into lactose and sialic acid. 3′-SL is an acidic HMO present in human milk. It consists of three monose units, sialic acid, galactose and glucose linked together. Lactose is a galactose unit linked to a glucose unit via a beta1,4linkage. A sialic acid unit is linked to a galactose unit of a lactose molecule via an alpha 2,3 linkage. 3′-SL is commercially available, for instance from Sigma-Aldrich or Christian Hansen-Jennewein.

Preferably, a nutritional composition according to the invention comprises at least 0.005 g 3′-SL per 100 ml, more preferably at least 0.01 g, even more preferably at least 0.02 g 3′-SL per 100 ml. Based on dry weight, the present nutritional composition preferably comprises at least 0.04% 3′-SL, more preferably at least 0.075 wt. %, even more preferably at least 0.15 wt. % 3′-SL. Based on energy, the present nutritional composition preferably comprises at least 0.008 g 3′-SL per 100 kcal, more preferably at least 0.015 g 3′-SL per 100 kcal, even more preferably at least 0.03 g 3′-SL per 100 kcal.

Preferably, a nutritional composition according to the invention comprises 0.005 g to 0.5 g 3′-SL per 100 ml, more preferably 0.01 g to 0.25 g, even more preferably 0.02 g to 0.1 g 3′-SL per 100 ml. Based on dry weight, the present nutritional composition preferably comprises 0.04 wt. % to 4 wt. % 3′-SL, more preferably 0.075 wt. % to 2.0 wt. % 3′-SL, even more preferably 0.15 wt. % to 0.75 wt. % 3′-SL. Based on energy, the present nutritional composition preferably comprises 0.008 to 0.75 g 3′-SL per 100 kcal, more preferably 0.015 to 0.04 g 3′-SL per 100 kcal, even more preferably 0.03 to 0.2 g 3′-SL per 100 kcal. A lower amount of 3′-SL will be less effective in stimulating growth of the bifidobacteria, whereas a too high amount may also increase the risk of osmotic diarrhea. This will counteract the beneficial effects of the mix on the intestinal health.

6′-Sialyllactose (6-′SL) is split by extracellular enzymes of B. bifidum into lactose and sialic acid. 6′-SL is an acidic HMO present in human milk. It consists of three monose units, sialic acid, galactose and glucose linked together. Lactose is a galactose unit linked to a glucose unit via a beta1,4linkage. A sialic acid unit is linked to a galactose unit of a lactose molecule via an alpha 2,6 linkage. 6′-SL is commercially available, for instance from Sigma-Aldrich or Christian Hansen-Jennewein.

Preferably, a nutritional composition according to the invention comprises at least 0.005 g 6′-SL per 100 ml, more preferably at least 0.01 g, even more preferably at least 0.02 g 6′-SL per 100 ml. Based on dry weight, the present nutritional composition preferably comprises at least 0.04 wt. % 6′-SL, more preferably at least 0.075 wt. % 6′-SL, even more preferably at least 0.15 wt. % 6′-SL. Based on energy, the present nutritional composition preferably comprises at least 0.008 g 6′-SL per 100 kcal, more preferably at least 0.015 g 6′-SL per 100 kcal, even more preferably at least 0.3 g 6′-SL per 100 kcal.

Preferably, a nutritional composition according to the invention comprises 0.005 g to 0.5 g 6′-SL per 100 ml, more preferably 0.01 g to 0.25 g, even more preferably 0.02 mg to 0.1 g 6′-SL per 100 ml. Based on dry weight, the present nutritional composition preferably comprises 0.04 wt. % to 4 wt. % 6′-SL, more preferably 0.075 wt. % to 2.0 wt. % 6′-SL, even more preferably 0.15 wt. % to 0.75 wt. % 6′-SL. Based on energy, the present nutritional composition preferably comprises 0.008 to 0.75 g 6′-SL per 100 kcal, more preferably 0.015 to 0.04 g 6′-SL per 100 kcal, even more preferably 0.03 to 0.15 g 6′-SL per 100 kcal. A lower amount of 6′-SL will be less effective in stimulating growth of the bifidobacteria, whereas a too high amount may also increase the risk of osmotic diarrhea. This will counteract the beneficial effects of the mix on the intestinal health.

Most preferably the nutritional composition comprises 2′-FL. 2′-FL was demonstrated to be very effective in the specific synbiotic mix, and it is also the most abundant milk oligosaccharide in human milk.

Not all HMOS will have the same effect as the four specific HMOS selected above. While lacto-N-tetraose (LNT) and lacto-N-neotetraose (LNnT) are abundant HMOs in human milk, these HMOS do not have a syntrophic effect. Many B. breve strains are already able to take up and metabolize these HMOS, so no syntrophic and synergistic effect is expected.

### Beta-Galacto-Oligosaccharides

Preferably beta-galacto-oligosaccharides (bGOS) are present in the nutritional composition, preferably bGOS with a DP of 4 or more. Preferably the bGOS has predominantly beta1,4linkages. bGOS is a non-digestible oligosaccharide (NDO). Thus in one embodiment, the nutritional composition according to the invention further comprises non-digestible galacto-oligosaccharide comprising beta1,4linkages, in particular beta1,4linkages between the galactose units, and having a degree of polymerization of at least 4.

A suitable way to form bGOS is to treat lactose with beta-galactosidases. Dependent on the specificity of the enzyme used, a galactose unit is hydrolysed from lactose and coupled to another lactose unit via a beta-linkage to form a trisaccharide. A galactose unit may also be coupled to another single galactose unit to form a disaccharide. Subsequent galactose units are coupled to form oligosaccharides. A suitable way to prepare beta1,4 GOS is by using the beta-galactosidase from Bacillus circulans or Cryptococcus laurentii. A commercially available source of bGOS is Vivinal® GOS from FrieslandCampina Domo (Amersfoort, The Netherlands). Vivinal® GOS comprises bGOS mainly with DP2-8 and with beta1,4linkages being more predominant. Van Leeuwen et al, 2014, Carbohydrate Res 400: 59-73 disclose that commercial Vivinal® GOS has about 1.5% Gal, 18.5% Glu, 42.5% DP2 (including the 21% lactose), 23.6% bGOS with DP3, 10.2% bGOS DP4, 3.0% bGOS DP5 and <0.5% bGOS DP6 and higher. Based on bGOS, so excluding lactose, galactose, and glucose, the amount of bGOS with DP4 or higher in Vivinal® GOS is thus about 22.4% based on total bGOS. Other suitable sources of bGOS with beta1,4linkages and comprising structures with DP4 or higher are Cup Oligo from Nissin Sugar, and galactan coming from potato tuber pectin (Megazyme Int). It is noted that in the context of the present invention oligosaccharides like beta1,3′-galactosyllactose, beta1,4′-galactosyllactose, beta1,6′-galactosyllactose may be present as part of the bGOS fraction and thus are not considered to be human milk oligosaccharides.

Preferably the nutritional composition comprises at least 250 mg bGOS per 100 ml, more preferably at least 400 mg even more preferably at least 600 mg per 100 ml. Preferably the nutritional composition does not comprise more than 2500 mg bGOS per 100 ml, preferably not more than 1500 mg, more preferably not more than 1000 mg per 100 ml. More preferably, the nutritional composition according to the present invention comprises bGOS in an amount of 250 to 2500 mg/100 ml, even more preferably in an amount of 400 to 1500 mg/100 ml, even more preferably in an amount of 600 to 1000 mg/100 ml.

Preferably the nutritional composition comprises at least 1.75 wt. % bGOS based on dry weight of the total composition, more preferably at least 2.8 wt. %, even more preferably at least 4.2 wt. % based on dry weight of the total composition. Preferably the nutritional composition does not comprise more than 17.5 wt. % bGOS based on dry weight of the total composition, more preferably not more than 10.5 wt. %, even more preferably not more than 7 wt. % based on dry weight of the total composition. The nutritional composition according to the present invention preferably comprises bGOS in an amount of 1.75 to 17.5 wt. %, more preferably in an amount of 2.8 to 10.5 wt. %, most preferably in an amount of 4.2 to 7 wt. %, all based on dry weight of the total composition. Preferably the nutritional composition according to the present invention comprises at least 0.35 g bGOS per 100 kcal, more preferably at least 0.6 g, even more preferably at least 0.8 g per 100 kcal. Preferably the nutritional composition does not comprise more than 3.7 g bGOS per 100 kcal, preferably not more than 2.5 g per 100 kcal, more preferably not more than 1.5 g per 100 kcal. More preferably, the nutritional composition according to the present invention comprises bGOS in an amount of 0.35 to 3.7 g per 100 kcal, even more preferably in an amount of 0.6 to 2.5 g per 100 ml, even more preferably in an amount of 0.8 to 1.5 g per 100 ml. Lower amounts result in a less effective composition, whereas the presence of higher amounts of bGOS may result in side-effects such as osmotic disturbances, abdominal pain, bloating, gas formation and/or flatulence.

In one embodiment, the nutritional composition according to the invention preferably comprises galacto-oligosaccharide comprising beta1,4linkages having a DP of at least 4 in an amount of at least 50 mg/100 ml. Preferably the nutritional composition comprises at least 80 mg bGOS having a DP of at least 4 per 100 ml, even more preferably at least 120 mg per 100 ml. Preferably the nutritional composition does not comprise more than 500 mg bGOS having a DP of at least 4 per 100 ml, preferably not more than 300 mg, more preferably not more than 200 mg. More preferably, the nutritional composition according to the present invention comprises bGOS having a DP of at least 4 in an amount of 50 to 500 mg/100 ml, even more preferably in an amount of 800 to 300 mg/100 ml, even more preferably in an amount of 120 to 200 mg/100 ml.

Preferably the nutritional composition comprises at least 0.35 wt. % bGOS with DP4 or higher based on dry weight of the total composition, more preferably at least 0.6 wt. %, even more preferably at least 0.8 wt. %, all based on dry weight of the total composition. Preferably the nutritional composition does not comprise more than 3.5 wt. % bGOS with DP4 or higher based on dry weight of the total composition, more preferably not more than 2 wt. %, even more preferably not more than 1.5 wt. %. The nutritional composition according to the present invention preferably comprises bGOS with DP4 or higher in an amount of 0.35 to 3.5 wt. %, more preferably in an amount of 0.6 to 2 wt. %, most preferably in an amount of 0.8 to 1.5 wt. %, all based on dry weight of the total composition.

Preferably the nutritional composition according to the present invention comprises at least 0.07 g bGOS with DP4 or higher per 100 kcal, more preferably at least 0.12 g, even more preferably at least 0.16 g per 100 kcal. Preferably the nutritional composition does not comprise more than 0.75 g bGOS with DP4 or higher per 100 kcal, preferably not more than 0.5 g per 100 kcal, more preferably not more than 0.3 g per 100 kcal. More preferably, the nutritional composition according to the present invention comprises bGOS with DP4 or higher in an amount of 0.07 to 0.75 g per 100 kcal, even more preferably in an amount of 0.12 to 0.5 g per 100 ml, even more preferably in an amount of 0.16 to 0.3 g per 100 ml. Lower amounts result in a less effective composition, whereas the presence of higher amounts of bGOS may result in side-effects such as osmotic disturbances, abdominal pain, bloating, gas formation and/or flatulence.

Preferably the nutritional composition according to the present invention also comprises fructo-oligosaccharides (FOS). Preferably the fructo-oligosaccharides have a DP or average DP in the range of 2 to 250, more preferably 2 to 100, even more preferably 10 to 60. Preferably the nutritional composition comprises long chain fructo-oligosaccharides (IcFOS), also referred to as non-digestible polyfructose, with an average DP of at least 11, more preferably at least 20. FOS suitable for use in the composition of the invention is also readily commercially available, e.g. RaftilineHP® (Orafti). Preferably the nutritional composition according to the present invention comprises at least 25 mg FOS, preferably polyfructose with an average DP of at least 11, more preferably of at least 20, per 100 ml, more preferably at least 40 mg even more preferably at least 60 mg per 100 ml. Preferably the composition does not comprise more than 250 mg FOS, preferably polyfructose with an average DP of at least 11, more preferably of at least 20, per 100 ml, more preferably not more than 150 mg per 100 ml and most preferably not more than 100 mg per 100 ml. The amount of FOS, preferably polyfructose with an average DP of at least 11, more preferably of at least 20, is preferably 25 to 250 mg per 100 ml, preferably 40 to 150 g per 100 ml, more preferably 60 to 100 g per 100 ml. Preferably the nutritional composition according to the present invention comprises at least 0.15 wt. % FOS, preferably polyfructose with an average DP of at least 11, more preferably of at least 20, based on dry weight, more preferably at least 0.25 wt. %, even more preferably at least 0.4 wt. %. Preferably the composition does not comprise more than 1.5 wt. % FOS, preferably polyfructose with an average DP of at least 11, more preferably of at least 20, based on dry weight of the total composition, more preferably not more than 2 wt. %. The presence of FOS, preferably polyfructose with an average DP of at least 11, more preferably of at least 20, together with bGOS shows a further improved effect on the microbiota and its SCFA production, which aid the restoration to a balanced microbiota after a disturbing event.

Preferably the present nutritional composition comprises a mixture of bGOS and FOS. Preferably the mixture of bGOS and FOS is present in a weight ratio of from 1/99 to 99/1, more preferably from 1/19 to 19/1, more preferably from 1/1 to 19/1, more preferably from 2/1 to 15/1, more preferably from 5/1 to 12/1, even more preferably from 8/1 to 10/1, even more preferably in a ratio of about 9/1. This weight ratio is particularly advantageous when the bGOS have a low average DP and FOS has a relatively high DP. Most preferred is a mixture of bGOS with an average DP below 10, preferably below 6, and FOS with an average DP above 7, preferably above 11, even more preferably above 20. Preferably FOS is polyfructose with an average DP of at least 11, more preferably with an average DP of at least 20.

Preferably the weight ratio of the sum of 2′-FL, 3-FL, 3′-SL and 6′-SL to bGOS is 1:1 to 1:40, more preferably 1:1.5 to 1:20.

Preferably the weight ratio of 2′-FL to bGOS is 1:1 to 1:40, more preferably 1:1.5 to 1:20.

Preferably the weight ratio of the sum of 2′-FL, 3-FL, 3′-SL and 6′-SL to bGOS to IcFOS is 1:(1-40):(0.1-4), more preferably 1:(1.5-20):(0.15-2.0).

Preferably the weight ratio of 2′-FL to bGOS to IcFOS is 1:(1-40):(0.1-4), more preferably 1:(1.5-20):(0.15-2.0). These ratios will improve an improved syntrophic effect of the mix of specific bifidobacteria and non-digestible oligosaccharides.

Preferably the weight ratio of the sum of 2′-FL, 3-FL, 3′-SL and 6′-SL to bGOS with DP4 or more is 1:0.25 to 1:10 more preferably 1:0.3 to 1:5.

Preferably the weight ratio of 2′-FL to bGOS with DP4 or more is 1:0.25 to 1:10, more preferably 1:0.3 to 1:5.

Preferably the weight ratio of the sum of 2′-FL, 3-FL, 3′-SL and 6′-SL to bGOS with DP4 or more to IcFOS is 1:(0.25-10):(0.1-4), more preferably 1:(0.3-5):(0.15-2.0).

Preferably the weight ratio of 2′-FL to bGOS with DP4 or more to IcFOS is 1:(0.25-10):(0.1-4), more preferably 1:(0.3-5):(0.15-2.0).

These ratios will improve an improved syntrophic effect of the mix of specific Bifidobacterium species and non-digestible oligosaccharides.

### Nutritional Compositions

The nutritional composition according to the present invention is not human milk. The nutritional composition is not mammalian milk. For sake of completeness, mammalian milk, or human milk, refers to milk as it naturally occurs, so it may also referred to an natural mammalian milk, or natural human milk. The nutritional composition according to the present invention is a synthetic formula. The nutritional composition according to the present invention is preferably for use in children, more preferably in infants or young children.

The present nutritional composition preferably comprises lipid, protein and carbohydrate and is preferably administered in liquid form. The present nutritional composition may also be in the form of a dry food, preferably in the form of a powder which is accompanied with instructions as to mix said dry food, preferably powder, with a suitable liquid, preferably water. The present nutritional composition may thus be in the form of a powder, suitable to reconstitute with water to provide a ready-to-drink nutritional composition, preferably a ready-to-drink infant formula, follow-on formula or young child formula, more preferably a ready-to-drink infant formula or follow-on formula. Powder is preferred, because it will improve the shelf life of a product containing the live bifidobacteria. The nutritional composition according to the invention preferably comprises other fractions, such as vitamins, minerals, trace elements and other micronutrients in order to make it a complete nutritional composition. Preferably infant formulae and follow-on formulae comprise vitamins, minerals, trace elements and other micronutrients according to international directives. Thus in one embodiment, the nutritional composition according to the invention any further comprises protein, carbohydrates, lipids, vitamins and minerals and is a liquid or a powder suitable to be reconstituted to a liquid, and the nutritional composition is preferably an infant formula, a follow on formula or a young child formula.

The present nutritional composition preferably comprises lipid, protein and digestible carbohydrate wherein the lipid provides 25 to 65% of the total calories, the protein provides 6.5 to 16% of the total calories, and the digestible carbohydrate provides 20 to 80% of the total calories. Preferably, in the present nutritional composition the lipid provides 30 to 55% of the total calories, the protein provides 7 to 9% of the total calories, and the digestible carbohydrate provides 35 to 60% of the total calories.

Preferably the present composition comprises at least one lipid selected from the group consisting of vegetable lipids. Preferably the present composition comprises a combination of vegetable lipids and at least one oil selected from the group consisting of fish oil, algae oil, fungal oil, and bacterial oil. The lipid of the present nutritional composition preferably provides 3 to 7 g per 100 kcal of the nutritional composition, preferably the lipid provides 3.5 to 6 g per 100 kcal. When in liquid form, e.g. as a ready-to-feed liquid, the nutritional composition preferably comprises 2.0 to 6.5 g lipid per 100 ml, more preferably 2.5 to 4.0 g per 100 ml. Based on dry weight the present nutritional composition preferably comprises 15 to 45 wt. % lipid, more preferably 20 to 30 wt. Preferably the present nutritional composition comprises at least one, preferably at least two lipid sources selected from the group consisting of rape seed oil (such as colza oil, low erucic acid rape seed oil and canola oil), high oleic sunflower oil, high oleic safflower oil, olive oil, marine oils, microbial oils, coconut oil, palm kernel oil.

The present nutritional composition preferably comprises protein. The protein used in the nutritional composition is preferably selected from the group consisting of non-human animal proteins, preferably milk proteins, vegetable proteins, such as preferably soy protein and/or rice protein, and mixtures thereof. The present nutritional composition preferably contains casein, and/or whey protein, more preferably bovine whey proteins and/or bovine casein. Thus in one embodiment the protein in the present nutritional composition comprises protein selected from the group consisting of whey protein and casein, preferably whey protein and casein, preferably the whey protein and/or casein is from cow's milk. Preferably the protein comprises less than 5 wt. % based on total protein of free amino acids, dipeptides, tripeptides or hydrolysed protein. The present nutritional composition preferably comprises casein and whey proteins in a weight ratio casein:whey protein of 10:90 to 90:10, more preferably 20:80 to 80:20, even more preferably 35:65 to 55:45.

The wt. % protein based on dry weight of the present nutritional composition is calculated according to the Kjeldahl-method by measuring total nitrogen and using a conversion factor of 6.38 in case of casein, or a conversion factor of 6.25 for other proteins than casein. The term ‘protein’ or ‘protein component’ as used in the present invention refers to the sum of proteins, peptides and free amino acids.

The present nutritional composition preferably comprises protein providing 1.6 to 4.0 g protein per 100 kcal of the nutritional composition, preferably providing 11.7 to 2.3 g per 100 kcal of the nutritional composition. A too low protein content based on total calories will result in less adequate growth and development in infants and young children. A too high amount will put a metabolic burden, e.g. on the kidneys of infants and young children. When in liquid form, as a ready-to-feed liquid, the nutritional composition preferably comprises 1.0 to 3.0 g, more preferably 1.0 to 1.5 g protein per 100 ml. Based on dry weight the present nutritional composition preferably comprises 8 to 20 wt. % protein, more preferably 8.5 to 11.5 wt. %, based on dry weight of the total nutritional composition.

The present nutritional composition preferably comprises digestible carbohydrate providing 5 to 20 g per 100 kcal, preferably 8 to 15 g per 100 kcal. Preferably the amount of digestible carbohydrate in the present nutritional composition is 25 to 90 wt. %, more preferably 8.5 to 11.5 wt. %, based on total dry weight of the composition. Preferred digestible carbohydrates are lactose, glucose, sucrose, fructose, galactose, maltose, starch and maltodextrin. Lactose is the main digestible carbohydrate present in human milk. The present nutritional composition preferably comprises lactose. Preferably the present nutritional composition does not comprise high amounts of carbohydrates other than lactose. Compared to digestible carbohydrates such as maltodextrin, sucrose, glucose, maltose and other digestible carbohydrates with a high glycemic index, lactose has a lower glycemic index and is therefore preferred. The present nutritional composition preferably comprises digestible carbohydrate, wherein at least 35 wt. %, more preferably at least 50 wt. %, more preferably at least 60 wt. %, more preferably at least 75 wt. %, even more preferably at least 90 wt. %, most preferably at least 95 wt. % of the digestible carbohydrate is lactose. Based on dry weight the present nutritional composition preferably comprises at least 25 wt. % lactose, preferably at least 40 wt. %, more preferably at least 50 wt. % lactose.

It is also important that the nutritional composition according to the present invention does not have an excessive caloric density, but still provides sufficient calories to feed the infant or young child. Hence, the liquid food preferably has a caloric density between 0.1 and 2.5 kcal/ml, more preferably a caloric density of between 0.5 and 1.5 kcal/ml, even more preferably between 0.5 and 0.8 kcal/ml, and most preferably between 0.65 and 0.7 kcal/ml.

### Application

The present nutritional composition is preferably an infant formula, a follow-on formula or a young child formula. Examples of a young child formula are toddler milk, toddler formula and growing up milk. More preferably the nutritional composition is an infant formula or a follow-on formula. The present nutritional composition can be advantageously applied as a complete nutrition for infants. An infant formula is defined as a formula for use in infants and can for example be a starter formula, intended for infants of 0 to 6 or 0 to 4 months of age. A follow-on formula is intended for infants of 4 or 6 months to 12 months of age. At this age infants start weaning on other food. A young child formula, or toddler or growing up milk or formula is intended for children of 12 to 36 months of age.

The present invention also concerns a method of providing nutrition to a child, comprising administering a nutritional composition according to the invention to the child. In other words, the nutritional composition according to the invention is preferably for use in providing nutrition to a child. The invention can also be worded as the use of a mix of Bifidobacterium species and at least one human milk oligosaccharide as defined according to the invention, for the preparation of a nutritional composition for providing nutrition to a child. A child is defined as a human with an age of 10 years or below. More preferably the nutritional composition according to the invention is for use in providing nutrition to a child with an age of 6 years or below, more preferably a young child with an age of 36 months or below, even more preferably an infant with an age of 12 months or below. Children have a less stable microbiota than adults and are therefore more susceptible to microbiota disruptive events. Children have less microbiota resilience, and after a disruptive event the microbiota is more at risk of not retuning back to the previous well-balanced state. This may have short- and long-term health effects. The younger the child, the less stable the microbiota.

In one embodiment the nutritional composition is for use in a human subject suffering from or at risk of intestinal microbial dysbiosis. Subjects at risk of intestinal dysbiosis are children, in particular young children, and especially infants.

It is known in the art that the microbiome of infants born preterm or via caesarean section is unstable and underdeveloped compared to term and vaginally born infants. These groups of infants therefore will benefit from the presently found combination of mix of Bifidobacterium species with specific HMO to address microbiota imbalance. Caesarean section born infants and infants treated with antibiotics suffer from the same problem of having a reduced and compromised microbiota, or microbial dysbiosis. In that respect antibiotic treatment can be seen as a model for born via caesarean section.

In one embodiment, human a subject suffering from or being at risk of intestinal microbial dysbiosis is a subject that was born preterm, a subject that was born via caesarean section, a subject that is or has been treated with antibiotics, a subject that is or has been at least partly breastfed by a woman taking antibiotics and an infant that is fully formula-fed with formula that do not contain non-digestible oligosaccharides. Preferably, the nutritional composition is for use in a subject, preferably a child, more preferably an infant that is or has been treated with antibiotics. Preferably the nutritional composition is for use in a young child or infant, more preferably an infant, that was born via caesarean section. Preferably the nutritional composition is for use directly or shortly after birth in an infant that is treated with antibiotics directly or shortly after birth, preferably in an infant that is born preterm and is treated with antibiotics directly or shortly after birth.

The nutritional composition according to the invention is preferably for use in preventing and/or treating intestinal microbial dysbiosis in a human subject suffering from or at risk of intestinal microbial dysbiosis, preferably selected from the group consisting of a subject that was born preterm, a subject that was born via caesarean section, a subject that is or has been treated with antibiotics, a subject that is at least partly breastfed by a woman taking antibiotics.

In other words, the present invention also concerns a method for preventing and/or treating intestinal microbial dysbiosis in a human subject suffering from or at risk of intestinal microbial dysbiosis, preferably selected from the group consisting of a subject that was born preterm, a subject that was born via caesarean section, a subject that is or has been treated with antibiotics, a subject that is at least partly breast fed by a woman taking antibiotics, comprising administering a nutritional composition according to the invention to said human subject.

The invention can also be worded as the use of a mix of Bifidobacterium species and at least one human milk oligosaccharide as defined according to the invention, for the preparation of a nutritional composition for preventing and/or treating intestinal microbial dysbiosis in a human subject suffering from or at risk of intestinal microbial dysbiosis, preferably selected from the group consisting of a subject that was born preterm, a subject that was born via caesarean section, a subject that is or has been treated with antibiotics, a subject that is at least partly breast fed by a woman taking antibiotics.

Preferably the (use in) preventing and/or treating intestinal microbial dysbiosis is directly or shortly after birth in an infant that is treated with antibiotics directly or shortly after birth, preferably in an infant that is born preterm and is treated with antibiotics directly or shortly after birth.

The specific combination of Bifidobacterium species and non-digestible oligosaccharides of the present invention was shown to have superior syntrophic effects on fermentation, growth, growth stimulatory effect on other bifidobacteria and anti-pathogenic properties. These effects were achieved by making optimal use of and sharing substrates, producing more and different beneficial metabolites in the supernatant, stimulating growth of other bifidobacteria and decreasing the growth of intestinal (opportunistic) pathogenic bacteria. Combinations that contained other bacterial species showed a synergistic effect to a lesser extent, did not show a synergistic effect or even showed an antagonistic effect. Using a model involving antibiotics for microbiota disturbances, it was found this specific combination of Bifidobacterium species and non-digestible oligosaccharides maintained these syntrophic properties under conditions present in the intestine in the presence of a microbiota that had been disturbed with an antibiotic treatment. This is indicative of the ability of the specific combination of Bifidobacterium species and specific non-digestible oligosaccharides to prevent or to treat intestinal microbial dysbiosis and increase the ability of the microbiota to return back to a well-balanced state after a disruptive event. Furthermore, it was found in faecal samples that was either compromised by antibiotic treatment or not compromised, the presence of the specific combination of Bifidobacterium species and specific non-digestible oligosaccharides showed a greater restorative effect in the compromised microbiota. Likewise, a greater improvement in the development of microbiota was observed in case of birth via caesarean section versus vaginal birth.

The nutritional composition of the present invention is preferably for use in improving the intestinal health in a child, preferably an infant. The nutritional composition of the present invention is preferably for use in preventing and/or treating intestinal microbial dysbiosis and/or for use in reducing intestinal pathogenic bacteria in a child, preferably an infant. The nutritional composition of the present invention is preferably for use in improving the intestinal health in a child, more preferably an infant, that was born via caesarean section. Preferably the nutritional composition of the present invention is for use in preventing and/or treating intestinal microbial dysbiosis and/or for use in reducing intestinal pathogenic bacteria in a young child or infant, more preferably an infant, that was born via caesarean section.

In other words, the present invention also concerns a method of improving the intestinal health in a child, preferably an infant, comprising administering a nutritional composition according to the invention to the child, preferably the infant. The present invention also concerns a method for preventing and/or treating intestinal microbial dysbiosis and/or for reducing intestinal pathogenic bacteria in a child, preferably an infant, comprising administering a nutritional composition according to the invention to the child, preferably the infant. The present invention also concerns a method for improving the intestinal health in a child, more preferably an infant, that was born via caesarean section, comprising administering a nutritional composition according to the invention to the child, preferably the infant. The present invention also concerns a method for preventing and/or treating intestinal microbial dysbiosis and/or for use in reducing intestinal pathogenic bacteria in a young child or infant, more preferably an infant, that was born via caesarean section, comprising administering a nutritional composition according to the invention to the young child or infant, preferably the infant, that was born via caesarean section.

The invention can also be worded as the use of a mix of Bifidobacterium species and at least one human milk oligosaccharide as defined according to the invention, for the preparation of a nutritional composition for improving the intestinal health in a child, preferably an infant. The invention can also be worded as the use of a mix of Bifidobacterium species and at least one human milk oligosaccharide as defined according to the invention, for the preparation of a nutritional composition for preventing and/or treating intestinal microbial dysbiosis and/or for reducing intestinal pathogenic bacteria in a child, preferably an infant. The invention can also be worded as the use of a mix of Bifidobacterium species and at least one human milk oligosaccharide as defined according to the invention, for the preparation of a nutritional composition for improving the intestinal health in a child, more preferably an infant, that was born via caesarean section. The invention can also be worded as the use of a mix of Bifidobacterium species and at least one human milk oligosaccharide as defined according to the invention, for the preparation of a nutritional composition for preventing and/or treating intestinal microbial dysbiosis and/or for use in reducing intestinal pathogenic bacteria in a young child or infant, more preferably an infant, that was born via caesarean section.

The present invention also concerns providing nutrition to a human subject suffering from or at risk of intestinal microbial dysbiosis, preferably selected from the group consisting of a subject that was born preterm, a subject that was born via caesarean section, a subject that is or has been treated with antibiotics, a subject that is at least partly breastfed by a woman taking antibiotics, a subject exposed to food or air pollution, a subject born from parents, in particular a mother, suffering from obesity, diabetes or allergy malnutrition, and a subject that received infant formula that did not contain human milk oligosaccharides, more preferably a subject selected from an infant or young child that was born via caesarean section or that is or has been treated with antibiotics.

The nutritional composition of the present invention is preferably for use in increasing the microbiota resilience against a microbiota disturbing event, wherein preferably the microbiota disturbing event is an antibiotic treatment. The nutritional composition of the present invention is preferably for use in restoring the intestinal microbiota that was exposed to a microbiota disturbing event, preferably wherein the microbiota disturbing event is an antibiotic treatment. The nutritional composition of the present invention is preferably for use in a faster restoring of the intestinal microbiota to its initial well-balanced state that was exposed to a microbiota disturbing event, preferably wherein the microbiota disturbing event is an antibiotic treatment. Preferably the use is in a child, more preferably a young child, even more preferably in an infant. In one embodiment, the microbiota disturbing event is an antibiotic treatment directly or shortly after birth, preferably an antibiotic treatment in an infant that is born preterm.

In other words, the present invention also concerns a method for
increasing the microbiota resilience against a microbiota disturbing event;
restoring the intestinal microbiota that was exposed to a microbiota disturbing event;
a faster restoring of the intestinal microbiota to its initial well-balanced state that was exposed to a microbiota disturbing event,
   wherein preferably the microbiota disturbing event is an antibiotic treatment, comprising administering a nutritional composition according to the invention to a subject in need thereof, preferably in a child, more preferably a young child, even more preferably in an infant.

The invention can also be worded as the use of a mix of Bifidobacterium species and at least one human milk oligosaccharide as defined according to the invention, for the preparation of a nutritional composition for i) increasing the microbiota resilience against a microbiota disturbing event, ii) restoring the intestinal microbiota that was exposed to a microbiota disturbing event, iii) a faster restoring of the intestinal microbiota to its initial well-balanced state that was exposed to a microbiota disturbing event, wherein preferably the microbiota disturbing event is an antibiotic treatment.

The nutritional composition of the present invention is preferably for use in alleviating the effects of antibiotic use on the intestinal microbiota.

In other words, the present invention also concerns a method for alleviating the effects of antibiotic use on the intestinal microbiota, comprising administering a nutritional composition according to the invention to a subject in need thereof.

The invention can also be worded as the use of a mix of Bifidobacterium species and at least one human milk oligosaccharide as defined according to the invention, for the preparation of a nutritional composition for alleviating the effects of antibiotic use on the intestinal microbiota.

The present invention also concerns aiding a healthy, preferably in terms of absolute and diverse, bifidogenic rich microbiota development, making the microbiota more resilient, for instance in an infant born by caesarian section, a preterm born infant or when the mother is treated with antibiotics.

In the context of the present invention, any reference to antibiotics preferably refers to oral antibiotics.

In the context of the present invention, directly or shortly after birth is within 10 days after birth.

The nutritional composition of the present invention is preferably for use in reducing the risk of occurrence, preventing and/or treating an intestinal infection, intestinal inflammation and/or diarrhea, preferably in a child, preferably in an infant.

In other words, the present invention also concerns a method for reducing the risk of occurrence, preventing and/or treating an intestinal infection, intestinal inflammation and/or diarrhea, comprising administering a nutritional composition according to the invention to a subject in need thereof, preferably to a child, preferably to an infant.

The invention can also be worded as the use of a mix of Bifidobacterium species and at least one human milk oligosaccharide as defined according to the invention, for the preparation of a nutritional composition for reducing the risk of occurrence, preventing and/or treating an intestinal infection, intestinal inflammation and/or diarrhea, preferably in a child, preferably in an infant.

Preferably the intestinal infection is bacterial intestinal infection. Preferably the diarrhea is acute diarrhea and/or antibiotic associated diarrhea. Preferably the intestinal inflammation is necrotizing enterocolitis.

A well balanced microbiota also affects the gut comfort and gut physiology. The nutritional composition of the present invention is preferably for use in reducing the risk of occurrence, preventing and/or treating colics and/or irritable bowel syndrome, preferably in a child, preferably in an infant.

A well balanced microbiota also affects the immune system and the occurrence or risk of occurrence of atopic disease. The nutritional composition of the present invention is preferably for use in reducing the risk of occurrence, preventing and/or treating allergy, atopic dermatitis, allergic rhinitis and allergic asthma, preferably in a child, preferably in an infant.

In the context of the present invention the term “prevention” means “reducing the risk of (occurrence)” or “reducing the severity of”. The term “prevention of a certain condition” also includes “treatment of a person at (increased) risk of said condition”.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### BRIEF DESCRIPTION OF THE FIGURES

FIG. 1 shows the glycoprofiles of supernatant of fermentation with GOS/FOS/2′-FL as carbohydrate source by individual strains and mixes.

FIG. 2 is a simplified plot of the Principal Component Analysis of the supernatants of individual strains and mixes grown on GOS/FOS/2′-FL.

### EXAMPLES

### Example 1: Fermentation of 2-Fucosyllactose by Single Strains and Mixes of Bifidobacterium Strains

### Materials and Methods

### Strains

The following Bifidobacterium strains were used. All strains were originally isolated from faeces of healthy infants.

| Species | beta1,4 endogalactanase | Company |
|---|---|---|
| B. bifidum CNCM I-4319 | − | Gervais Danone |
| B. breve C50 | ++ | Gervais Danone |
| B. breve M-16V | − | Morinaga |
| B. longum BB536 | − | Morinaga |
| B. infantis BB-02 | + | Christian Hansen |

The strains (B. breve C50, B. breve M-16V and B. bifidum CNCM I-4319) were transformed by pSH71-based (or pRB1 based) plasmids carrying resistance to different antibiotics using electroporation with the following plasmids.

| Strain | Plasmid | Replicon | Antibiotic resistance |
|---|---|---|---|
| B. breve C50 | pBC1 | pAM5 | Tetracycline (Tet) |
| B. bifidum CNCM | pDM1 | pBC1 | Spectinomycin (Spec) |
| I-4319 | | | |
| B. breve M-16V | pNZ44St | pSH71 | Streptomycin (Strep) |

To allow strain specific enumeration by plate count methods, the bifidobacterial strains were equipped with antibiotic resistance markers. To this pDM1 (O'Connell Motherway M et al. (2014) PLoS ONE 9(4): e94875. doi:10.1371/journal.pone.0094875, pNZ44St (UCC) and pAM5 (Alvarez-Martin et al, (2008), Improved Cloning Vectors for Bifidobacteria, based on the Bifidobacterium catenulatum pBC1 Replicon, AEM, p. 4656-4665, doi:10.1128/AEM.00074-08) were transformed to B. bifidum CNCM I-4319, B. breve M16-V and B. breve C50, respectively using established electroporation methods (Hoedt E. C., et al (2021) Bifidobacterium Transformation. In: van Sinderen D., Ventura M. (eds) Bifidobacteria. Methods in Molecular Biology, vol 2278. Humana, New York, NY. https://doi.org/10.1007/978-1-0716-1274-3_2). TOS-mup agar plates containing 100 μg/ml Spectinomycin (pDM1), 100 μg/ml Streptomycin (pNZ44st), 5 μg/ml Tetracyclin (pAM5) were used for strain-specific enumeration, and on non-selective TOS-mup agar plate to determine total bifidobacterial counts.

Alternative to antibiotic resistance, bifidobacteria can be plated on an agar plate selective for bifidobacteria and the number of individual colonies can randomly picked and be identified on strain level by molecular fingerprint techniques known in the art.

### Bifidobacterium Growth Kinetics on 2′-FL

Bifidobacterium strains were anaerobically cultured at 37° C. on trans-galacto-oligosaccharide agar (TOS-Propionate Agar (Base), Merck, Darmstadt, Germany) supplemented with bifidobacterial selective component MUP (MUP selective supplement, HC735221, Merck KGaA, Darmstadt, Germany). Anaerobic conditions were applied by cultivation in an anaerobic cabinet with 90% N2, 5% H2, 5% CO2 atmosphere. Bifidobacterium strains were routinely streaked from their master seed lot (−80° C. stock) on TOS-Mup agar and were incubated anaerobically at 37° C. for two days. Single colonies of each isolate were subsequently incubated overnight (approx. 18 hours) at 37° C. in (30 ml) TOS-Mup anaerobe broth. With this preculture the overnight pre-fermentations were inoculated. The inoculum volume of the pre-fermentation was 10% based on end volume of the culture volume. For example 25 ml inoculum is added to 225 ml modified BASE-Mup broth or Colonic Growth Medium. BASE-Mup broth is a specific optimized growth medium for bifidobacterial, while the Colonic Growth Medium was used to mimic more situation in colon of infants. As carbon source for the preculture 0.5 g L-1 was used.

For main fermentations a stock solution of 2′-fucosyllactose (Jennewein Biotechnology GmbH, Germany) was filter sterilized and added to either heat sterilized BASE-Mup medium or Filter sterilized Colonic Growth Medium to an end concentration of 2.5 wt/vol %. The pH of this medium was set at 6.5 unless indicated otherwise.

The composition of BASE-Mup broth was as follows: casein trypton (10 g L-1), yeast extract (1.0 g L-1), (NH₄)2SO₄ (3 g L-1), KH₂PO₄ (3 g L-1), K₂HPO₄ (4.8 g L-1), MgSO₄·7H₂O (0.2 g L-1) and L-cysteine hydrochloride (0.5 g L-1). The composition of Colonic Growth Medium was as follows: tryptone 10 g/L, casein 1 g/l, yeast extract 1 g/L, 2 g/L K₂HPO₄, 3.2 g/LNaHCO₃, 4.5 g/L NaCl, 3 g/L (NH₄)₂SO₄, 0.5 g/L MgSO₄·7H₂O, 0.5 g/L Cysteine·HCl, 0.4 g/L CaCl₂·2H₂O, 0.005 g/l FeSO₄·7H₂O, 0.01 g/l Haemin, 2 ml/l mineral solution 2 ml/l (containing per L: 500 mg EDTA, 200 mg FeSO₄·7H₂O, 10 mg ZnSO₄·7H₂O, 3 mg MnCl₂·7H₂O, 30 mg H₃BO₃, 20 mg CoCl₂·6H₂O, 1 mg CuCl₂·2H₂O, 2 mg NiCl₂·6H₂O, 3 mg NaMoO₄·2H₂O, 7.5 mg NaSeO³), 1.4 ml/l vitamin solution (containing per 11 g menadione, 2 g biotin, 2 g pantothenate, 10 g nicotinamide, 0.5 g cobalamine, 4 g thiamine, 5 g p-aminobenzoic acid; filter-sterilized.

The overnight pre-fermentations of the individual strains were performed anaerobically (90% N2, 5% H2, 5% CO2 headspace) in the DASGIP Parallel Bioreactor Systems (DASGIP Information and Process Technology GmbH, Julich, Germany) pH-control (pH 6.5) was achieved by dosing 5M NaOH.

Each fermenter of the main fermentations has a working volume of 250 ml and was inoculated with overnight precultures of the single strains or in case of the mixes of bifidobacterial strains equal ratios of the individual bifidobacterial in the mix were used so that the start optical density at 600 nm (OD) of the fermentation was 0.15. So with a mix of three strains the inoculum was made by adding of each strain such an amount that the starting OD in main fermentor was 0.05 OD for each of the three strains and was 0.15 in total. Growth was monitored for 24 h and during the fermentations samples were taken for further analysis like enumeration of individual species by spot-plating on selective TOS-propionate-agar plates, and glyco-profile analysis, or other analysis as described below. Samples for glycoprofiling were centrifuged for 10 min at 4250 RPM after which the supernatants were stored at −20° C. until analysis.

The viable count of each strain was determined by using the spot-plating method on TOS-Mup agar plates containing either 3 μg/ml tetracycline (87128, Sigma-Aldrich, St. Louis, Missouri, USA) for B. breve C50 pAM5-Tet CFU count, 2 μg/ml chloramphenicol (C0378, Sigma-Aldrich, St. Louis, Missouri, USA) for B. bifidum CNCM 1-4319 pNZ123-Cm CFU count or 50 μg/ml spectinomycin (S4014, Sigma-Aldrich, St. Louis, Missouri, USA) for B. breve M-16V pDM1-Spec CFU count or no other antibiotic for CFU count of total bifidobacterial (TotBif). A 5-fold serial dilution in a 96-wells plate was made from each sample in buffered peptone water with 0.05% cysteine (rBPW). 5 μl of each well (dilution) was spotted in duplicate on the selective plates, incubated for 48 hours anaerobically after which the colonies per spot were counted and CFU now could be calculated per strain per time point in the mix.

### Results

The results for NaOH consumption are shown in Table 1.

TABLE 1
NaOH consumed at t = 24 h (in ml of 5M solution) upon fermentation
of 2′-FL in BASE-Mup medium by single strains or mixes of strains
at pH 6.5. For a mix of strains the ‘Relative NaOH cons. vs single
strain (%)’ is compared to the best performing single strain of that mix.

| | | | | | | Relative NaOH |
|---|---|---|---|---|---|---|
| | | B. breve | B. breve | | NaOH | cons. vs single |
| B. infantis | B. bifidum | C50 | M-16V | B. longum | (mmol/l) | strain (%) |
| X | | | | | 278 | 100 |
| | X | | | | 232 | 100 |
| | | X | | | 0 | 0 |
| | | | X | | 0 | 0 |
| | | | | X | 0 | 0 |
| | | | X | X | 0 | 0 |
| | X | | X | X | 244 | 105* |
| X | X | | | | 273 | 98 ¹⁾ |
| X | | X | | | 209 | 76 ²⁾ |
| | X | X | | | 292 | 126** |
| | X | | X | | 246 | 106* |
| | X | X | X | | 286 | 123** |
| X | X | X | | | 279 | 100** ³⁾ |
| | X | | | X | 220 | 95 ¹⁾ |

*Mix according to the invention;
**Preferred mix according to the invention
¹⁾ No synergy on total NaOH consumption;
²⁾ Antagonism on NaOH consumption;
³⁾ No antagonism anymore

Based on the NaOH consumption at t=24 h (indicative for total acid produced upon fermentation) it can be concluded that B. infantis and B. bifidum could ferment 2′-FL as a single strain and the B. breve strains or B. longum could not ferment 2′-FL as single strain.

The combination of B. infantis and B. bifidum did not show an improved effect on NaOH consumption compared to the single strains.

When a combination of B. infantis with B. breve C50 was tested, the total amount of NaOH consumed with the mix was 25% decreased compared to the single B. infantis strain, indicative of an antagonistic effect. This was also the case when a mix of B. infantis and B. breve M-16V was tested (data not shown). Interestingly, when a mix of B. infantis, B. breve C50 and B. bifidum was tested, an NaOH consumption was observed which was similar to the B. infantis strain alone (which had a higher NaOH consumption that the B. bifidum strain alone) and this indicates that the antagonistic effect between B. breve and B. infantis has been alleviated when B. bifidum is also present in the mix.

The total amount of NaOH consumed was higher than the single strains only in specific mixes of Bifidobacterium strains, and only with mixes containing a combination of B. bifidum and B. breve. A mix of B. breve M-16V with the B. bifidum strain or a mix of B. breve M-16V with the B. bifidum strain and B. longum strain showed a higher NaOH consumption than that of B. bifidum alone (6% and 5% respectively). Also a mix of B. breve C50 with the B. bifidum strain showed a higher NaOH consumption than that of B. bifidum alone this was about 26% more NaOH consumed than could be expected based on the single B. bifidum strain. The same was found with a mix of B. breve C50, B. breve M-16V and B. bifidum. These results are indicative of synergy.

Besides the total amount of NaOH consumed, the rate of NaOH consumption during the logarithmic growth phase was higher in the mixes with each of both or both B. breve strains and B. bifidum compared to the single B. bifidum strain alone, and reduced in the combination of B. breve and B. infantis compared to the B. infantis strain alone (data not shown). The rate of NaOH consumption was slightly increased in the mix of B. breve, B. infantis and B. bifidum, indicating that not only the antagonistic effect between B. breve and B. infantis was alleviated, but even a small synergistic effect was observed (data not shown).

This effect on NaOH consumption was tested at pH 6.5 and 5.5. At lower pH the synergistic effect was even more pronounced. Table 2 shows the NaOH consumption at pH 5.5 and pH 6.5 in Colonic Growth Medium, t=24 h, of single strains and mixes. A pH of 5.5 is representative of the lumen of the colon of breast-fed healthy infants.

TABLE 2
NaOH consumed (in mmol/l) and relative amount of NaOH consumed
upon fermentation of 2′-FL by single strains or mixes
of strains at pH 5.5 and 6.5 t = 24 h (in Colonic Growth
Medium). For a mix of strains the ‘Relative (%)’ is
compared to the best performing single strain of that mix.

| | NaOH | Relative | NaOH | Relative |
|---|---|---|---|---|
| | (mmol/l) | (%) | (mmol/l) | (%) |
| strain | pH 5.5 | pH 5.5 | pH 6.5 | pH 6.5 |
| B. bifidum | 82 | 100 | 200 | 100 |
| B. breve C50 | — | — | — | — |
| B. breve M-16V | — | — | — | — |
| B. bifidum + B. breve C50 | 182 | 222 | 248 | 124 |
| B. bifidum + B. breve C50 + | 206 | 251 | 292 | 146 |
| B. breve M16V. | | | | |

Growth data as observe by increase of OD600 confirm that the single strains of B. breve are not able to grow on 2′-FL as sole carbon- and energy source, both at pH 6.5 and 5.5. B. infantis and B. bifidum alone, or the mixes containing these strains show a high end OD at pH 6.5 (data not shown).

At pH 5.5 the single B. bifidum strain did not grow towards a high end OD, but in the mix with B. breve C50 and M-16V the final OD reached was almost 5-fold higher. This again is in accordance with the NaOH consumption data and indicative of a synergistic or syntrophic effect in the mix of strains which is especially prominent at pH 5.5, a pH representative of the lumen of the colon of fully breast-fed healthy infants.

Plate counting indicated that in the mix all 3 strains were able to grow individually (table 3).

TABLE 3
Increase of bacterial concentration compared to the inoculum
(delta = measured cfu/ml − inoculum cfu/ml), of strains
added as single strains or present in a mix at mid logarithmic
phase (t = 5 h) and end logarithmic phase (t = 9
h) at pH 5.5 or 6.5. Fold increase is the ratio of the delta
(cfu/ml) divided by the bacterial concentration in the inoculum (cfu/ml).

| | | | t = 5 h | t = 9 h | t = 5 h | t = 9 h |
|---|---|---|---|---|---|---|
| | strain | | pH 6.5 | pH 6.5 | pH 5.5 | pH 5.5 |
| Single | B. bifidum | Delta cfu/ml | 4.6.10⁶ | 7.7.10⁸ | 2.7.10⁷ | 3.3.10⁷ |
| strains | | Fold increase | 10.7 | 17.4 | 2.5 | 2.9 |
| | B. breve | Delta cfu/ml | — | — | — | — |
| | C50 | Fold increase | — | — | — | — |
| | B. breve | Delta cfu/ml | — | — | — | — |
| | M-16V | Fold increase | — | — | — | — |
| Mix | B. bifidum | Delta cfu/ml | 2.2.10⁸ | nd | 3.3.10⁷ | 1.9.10⁷ |
| | | Fold increase | 18.9 | nd | 3.8 | 2.3 |
| | B. breve | Delta cfu/ml | 1.1.10⁹ | 1.5.10⁹ | 4.6.10⁸ | 1.6.10⁹ |
| | C50 | Fold increase | 20 | 27 | 16.3 | 52.5 |
| | B. breve | Delta cfu/ml | 9.0.10⁸ | 1.4.10⁹ | 4.9.10⁸ | 1.7.10⁹ |
| | M-16V | Fold increase | 23.1 | 35.6 | 18.9 | 62.1 |
| | | Fold increase | 62 | >63 | 39 | 117 |
| | | total | | | | |

nd: no data

B. bifidum, at pH 6.5, grew well and within 5 hours the cfu/ml was 10.7 times higher as provided with the inoculum. When grown in combination with the two B. breve strains the increase was even higher (18.9-fold). The B. breve strains, not able grow on 2′-FL alone, showed in a mix with the B. bifidum strain a very strong growth stimulation (20×). At lower pH, the B. bifidum did not grow well, but B. bifidum was still very well capable in stimulating the B. breve strains. The pH has less an effect on the two B. breve strains in combination with the B. bifidum.

### Example 2: Fermentation of a Mix of 20-Fucosyllactose and Galacto-Oligosaccharides and Fructo-Oligosaccharides by Single Strains and Mixes of Bifidobacterium Strains

### Introduction

The synergy of a mix of B. bifidum CNCM I-4319, B. breve C50 and B. breve M-16V when grown on a mixture of GOS/FOS/2′-FL was examined. To do so, a similar experiment was performed as in example 1, except that as carbon- and energy source a mixture of GOS/FOS/2′-FL was used. GOS/FOS/2′-FL was used in a wt/wt/wt ratio of 8:1:1 and at a 2.5 wt % final concentration. As a source of GOS Vivinal® GOS was used (Friesland Campina), as a source of FOS RaftilinHP (Orafti) The 2.5% carbohydrate concentration included the non-GOS carbohydrates (lactose, glucose and galactose) that are present in Vivinal® GOS. Experiments were performed at pH 6.5, unless indicated otherwise.

### Results

In Table 4 the final NaOH consumption is given. Compared to example 1 the final NaOH consumed was more comparable for the mixes of strains and the single strains because GOS/FOS was present in a relative high amount and most bacterial strains can grow well on these oligosaccharides. Nonetheless, the highest amount of NaOH consumed was again obtained with the mixes that contain B. bifidum and B. breve, in particular B. bifidum+B. breve C50, the mix of B. bifidum+B. breve C50+B. breve M16-V and the mix B. bifidum+B. breve C50+B. infantis.

TABLE 4
NaOH consumed upon fermentation of GOS/FOS/2′-FL in BASE-Mup medium
by single strains or mixes of strains at t = 24 h

| | | | | | | Relative NaOH cons. |
|---|---|---|---|---|---|---|
| | | B. breve | B. breve | | NaOH cons. | vs single strain |
| B infantis | B. bifidum | C50 | M-16V | B. longum | pH 6.5 (mM) | B. bifidum (%) |
| X | | | | | 280 | |
| | X | | | | 262 | 100 |
| | | X | | | 270 | |
| | | | X | | 252 | |
| | | | | X | 246 | |
| | X | | X | X | 295* | 113 |
| | X | | | X | 270 | 103 |
| | | | X | X | 252 | 100¹ |
| | X | X | | | 300** | 115 |
| | X | | X | | 284* | 108 |
| | X | X | X | | 290** | 111 |
| X | X | X | | | 280** | 107 |

*Mix according to the invention;
**Preferred mix according to the invention
¹vs single B. breve M 16V

TABLE 5
NaOH consumed (mmol/l) upon fermentation of GOS/FOS/2′-FL
in Colonic Growth Medium by single strains or mixes
of strains at pH 5.5 and 6.5 t = 24 h ml)

| | GOS/FOS/2′-FL | GOS/FOS/2′-FL |
|---|---|---|
| | pH 5.5 | pH 6.5 |
| B. bifidum | 72 | 276 |
| B. breve C50 | 258 | 318 |
| B. breve M-16V | 176 | 230 |
| B. breve C50 + B. bifidum | 280 | 359 |
| B. breve M16V + B. bifidum | 246 | 318 |
| B. bifidum, B. breve C50 + | 270 | 348 |
| B. breve M-16V. | | |

The effect of the pH on the syntrophic effect is shown in Table 5. Based on total amount of NaOH consumed, B. bifidum alone did not ferment the GOS/FOS/2′-FL very well at pH 5.5, compared to pH 6.5. At each pH values the highest amount of NaOH consumed was observed with the mix of B. bifidum, +B. breve C50 and B. bifidum+B. breve C650+M-16V and the amount was higher than that observed with single strains.

When looking at the OD growth data, at pH 6.5 a similar end OD is reached in the stationary phase, but a slightly faster growth rate was observed in the mix B. bifidum, B. breve C50 and M-16V (faster OD increase in the logarithmic phase) compared with the 3 single strains (data not shown).

Plating of the antibiotic resistant strains on antibiotic agar plates it could be determined that all 3 individual strains were present and growing in the mix, except that no increase in cfu was observed for B. bifidum at pH 5.5 in the mix and as single strain (data not shown).

### Example 3: Formation of Fermentation Products During the Fermentation Experiments of Example 1 and 2

### Material and Methods

### Glycoprofiling

Samples of the culture supernatant were taken during the fermentation experiments as described in examples 1 and 2. Samples for glycoprofiling taken during the fermentation were heated at 95° C. for 15 minutes to inactivate any enzymes and bacteria that were possibly still present. Samples were centrifuged for 10 min at 3250 g. After that, supernatants were filtered using a low-binding filter column (0.22 μm, MILLEX GV Durapore PVDF membrane, Merck, Darmstadt, Germany) and the samples were stored at −20° C. until analysis.

The glycoprofiling analysis was conducted using High Performance Anion Exchange Chromatography—Pulsed Electrochemical Detection (HPAEC-PED) (Dionex-HPLC equipment ICS5000, Thermo Fisher Scientific Inc, Waltham, USA) where a PA200 column was used (Dionex™ CarboPac™ 4*250 nm, P/N 43055, Thermo Fisher Scientific Inc, Waltham, USA). The principle is anion is exchanged where low pH gives carbohydrates a negative charge. Carbohydrates are then bond to the column after which elution of these proteins takes place making use of an eluent with a low hydroxide concentration and sodium acetate gradient. Detection is done with Pulsed Electrochemical Detection. Sugars are oxidized at a gold electrode surface which causes a current which is detected. The gold electrode surface is cleaned and activated with a cyclic current pulse train which is specific for carbohydrate detection. Elution is in order of amount of charge but also size and shape play a role in the retention of the component. As internal standard arabinose was used. Methods to determine the glycoprofile are known in the art. An examples is Finke et al, 2002, J. Agric. Food Chem. 2002, 50: 4743-4748.

### Metabolomics by 1H NMR Spectroscopy

For a selected amount of fermentation supernatant samples of t=9 or t=24 h, the metabolomics was analysed by 1H NMR spectroscopy. For GOS/FOS/2′-FL fermentations the individual strains and complete mixes were tested. For 2′-FL all samples of these time-points were analysed.

1H NMR spectroscopy measures protons (H) on metabolites and provides semi-quantitative and structural information on a diverse range of metabolite classes (sensitivity is in the μM range). Reproducibility, robustness and simple sample preparation are major strengths of NMR spectroscopy and make this technique particularly ideal for studying large-scale sample sets (>1000 samples) Samples were analyzed were analyzed by 1H NMR spectroscopy. For the culture supernatants the B.I.QUANT-UR method was used (Bruker BioSpin 08/2019 T165319) to quantify 50 known compounds from their NMR spectra. PCA models were built on the metabolic profiles (Caspani G, et al 2021, Metabolomic signatures associated with depression and predictors of antidepressant response in humans: A CAN-BIND-1 report. Commun Biol. 22; 4(1):903. doi: 10.1038/s42003-021-02421-6. PMID: 34294869; PMCID: PMC8298446.)

### Results

Glycoprofiles of Supernatant Fermentation with 2′-FL as Carbohydrate Source

For the fermentation experiments of example 1 the kinetics of sugar consumption and release in time was assessed by measuring glycoprofiles of the supernatant. From the glycoprofiles of the supernatants of single strains and mixes of strains grown on 2′-FL as single carbon and energy source it could be deduced that the B. infantis and B. bifidum strains consumed 2′-FL differently. The glycoprofile of the single B. infantis strain showed little to no formation of intermediate metabolites (such as fucose, lactose, glucose, galactose). Some fucose appeared in the supernatant, but then was decreasing again from t=7 h onwards. In contrast, in the supernatant with the B. bifidum strain the metabolites fucose and lactose appeared faster and in a higher amount. Lactose in turn disappeared and its degradation products glucose and galactose appeared, of which galactose was more slowly consumed than glucose. Fucose was not consumed by the B. bifidum strain.

The mix of B. longum, B. bifidum and B. breve M-16V showed an interaction. The formed lactose was consumed. The rate of lactose consumption was in the mix faster than by B. bifidum alone. Fucose was consumed by B. breve M16-V, as B. longum cannot utilize fucose.

The mixes comprising B. bifidum, B. breve C50 and as third strain B. infantis or B. breve M16-V showed the faster disappearance of 2′-FL and fucose in the supernatant. Also the intermediate metabolites fucose and lactose were degraded faster and more complete, compared to B. infantis alone or B. bifidum alone.

Metabolomics by 1H NMR Spectroscopy of the Fermentation with 2′-FL as Carbohydrate Source

Forty metabolites were found and assessed, which partly relate to carbohydrates, their hydrolysis products and the metabolites produced upon fermentation, including 2′-FL, fucose, lactose, galactose, glucose, acetic acid, lactic acid, formic acid, succinate, fumarate, ethanol, 1,2 propanediol, acetoin, indole-3-lactic acid.

Methods for performing metabolomics are known in the art. An example is Spitzer et al. iScience. 2021 Sep. 10; 24(10):103113. doi: 10.1016/j.isci.2021.103113. PMID: 34611610; PMCID: PMC8476651 and Beckonert, et al. Nat Protoc 2, 2692-2703 (2007). https://doi.org/10.1038/nprot.2007.376.

Patterns could be established, and relating to the amount of organic acids formed the results were very much in accordance with the NaOH consumption data and with the highest amount of acids formed with the mixes of the invention, and the lowest in the antagonistic mix of B. breve and B. infantis.

The main organic acid formed was acetic acid, but also lactic acid was present in substantial amounts. Related to the amounts of 2′-FL, lactose, galactose, glucose and fucose the results were very much in accordance with the glycoprofiling. These metabolites were indicative of a poor lactose or poor fucose metabolism.

Interestingly, B. bifidum as single strain did not produce 1,2 propanediol after fucose was split off from 2′-FL by the external alpha-L-fucosidase activity, but in the mix with B. breve 1,2 propanediol was formed, for all the three mixes tested (B. breve C50+B. bifidum+B. breve M-16V, B. breve C50+B. bifidum+B. infantis, B. breve M-16V+B. bifidum+B. longum) indicating that the fucose was catabolized into 1,2 propanediol, due to syntrophic interactions. 1,2 propanediol can serve as substrate for other beneficial bacteria in the microbiota. Acetoin was observed in low amounts with the single strain of B. bifidum, but was higher with the three mixes tested. Acetoin, a non-toxic pH-neutral overflow metabolite, is formed from acetate when extracellular acetate levels rise to high levels and it can be used as used as a carbon source in later growth stages. Acetoin is linked to the fucose metabolism.

Glycoprofiles of Supernatant of Fermentation with GOS/FOS/2′-FL as Carbohydrate Source

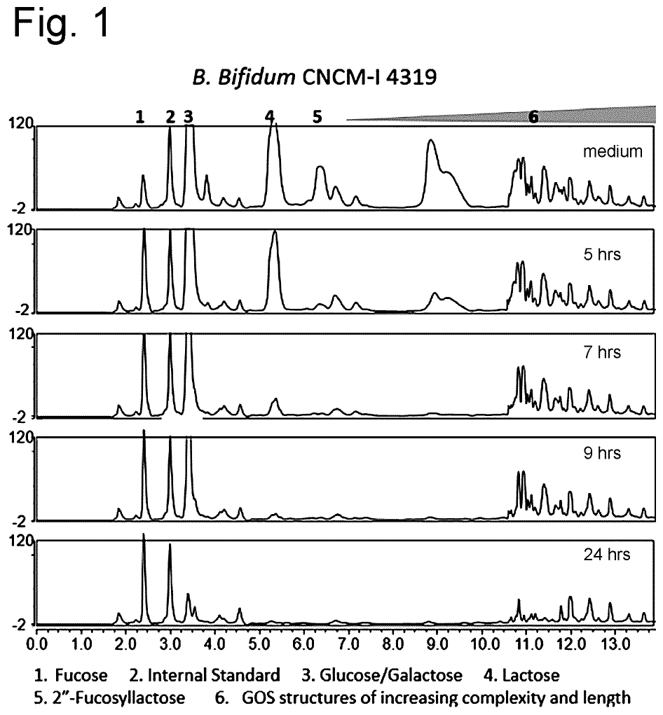

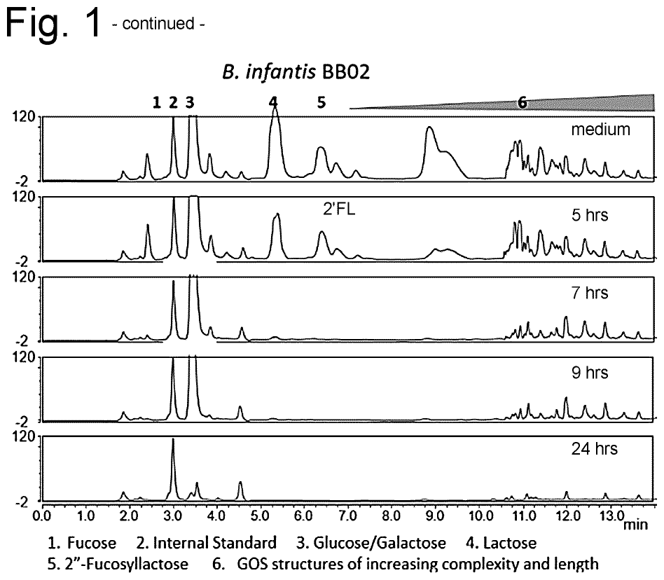

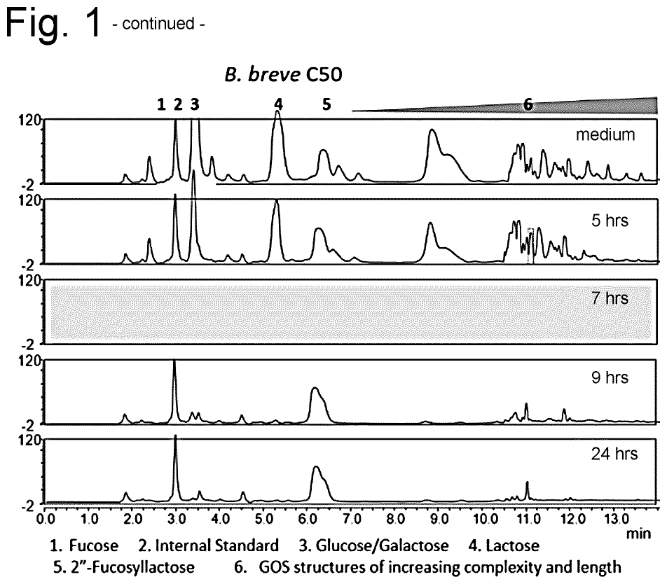

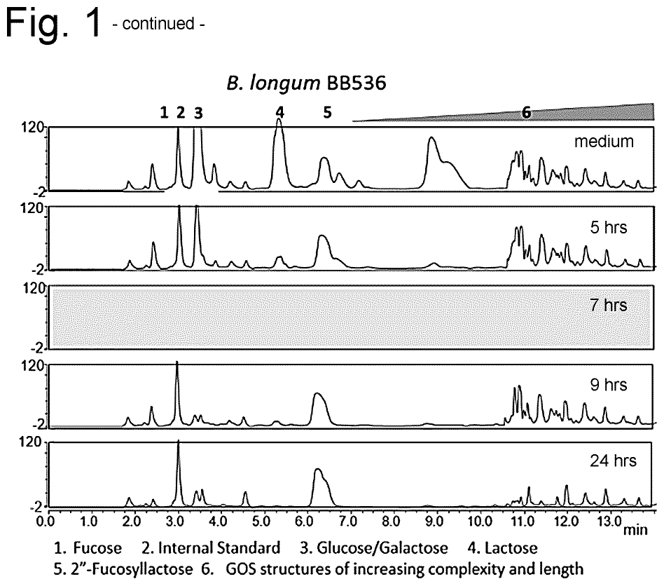

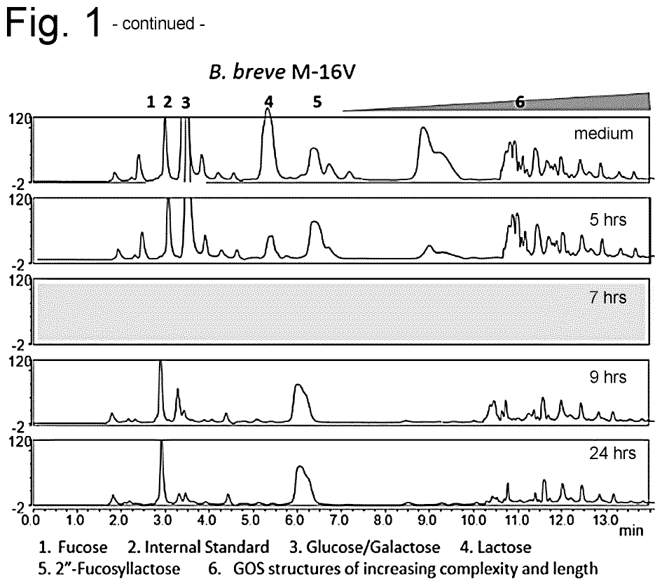

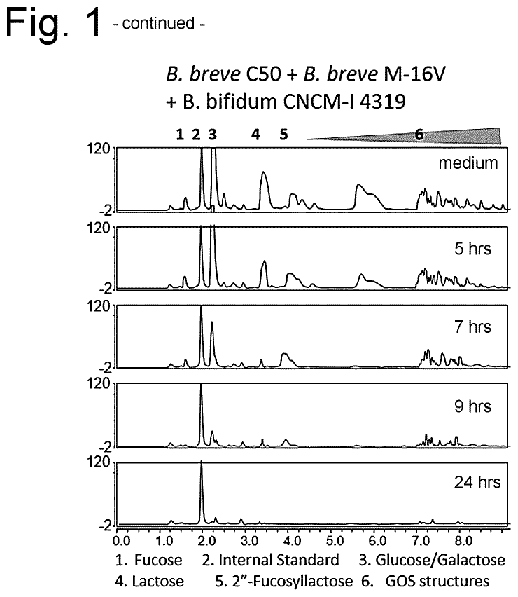

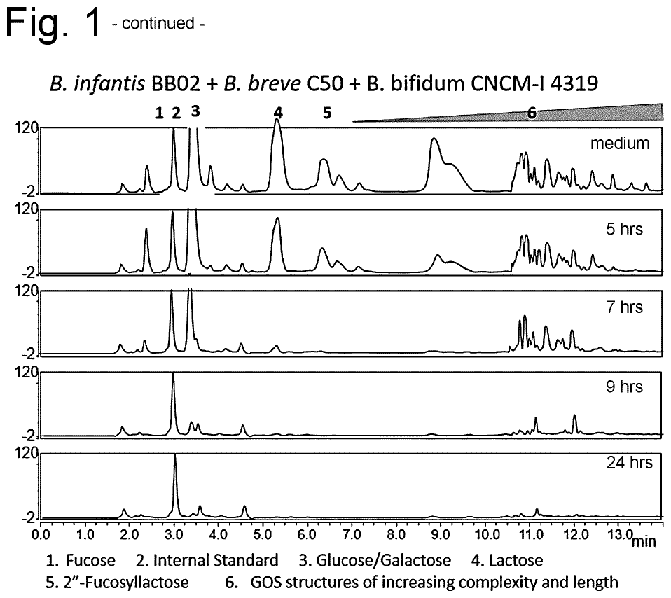

Also for the fermentation experiments of example 2 glycoprofiles were obtained during fermentation. The results are shown in FIG. 1. When looking at single strains it appeared that the 2′-FL was not consumed by the B. breve strains and B. longum and also no fucose was formed. 2′-FL was consumed by the single B. bifidum and B. infantis, and in case of B. bifidum fucose was formed and not further degraded, and higher levels of glucose and galactose remained during the first 9 hours. In case of single strain of B. infantis only very little fucose was found in the supernatant. Indicating intra-cellular alpha-L-fucosidase activity.

Lactose and short chain bGOS were consumed by all strains. Only B. breve C50, after consuming the short chain bGOS, consumed the bGOS with a higher DP, forming a new peak, likely beta1-4 linked galactotriose, main product of the endogalactanase activity, at 5 hours, which subsequently disappeared.

The mix of B. longum, B. bifidum and B. breve M-16V was able to split the 2′-FL into fucose and lactose. The lactose was immediately consumed, and the fucose was consumed slowly. When compared with mixes containing both B. bifidum and B. breve C50 this mix of B. longum, B. bifidum and B. breve M-16V was slightly less efficient in metabolizing the carbohydrates. Part of the bGOS structures, the higher DP structures, were not metabolized and remained in the supernatant. In contrast, the mix of B. bifidum, B. breve C50 and B. breve M-16V and the mix of B. bifidum, B. breve C50 and B. infantis were very efficient in syntrophic consumption of GOS/FOS/2′-FL including the from higher DP derived beta1-4 linked galactotriose due to the endogalactanase activity of B. breve C50

Metabolomics by 1H NMR Spectroscopy of the Fermentation with GOS/FOS/2′-FL as Carbohydrate Source

Forty metabolites were found and assessed, which partly relate to carbohydrates, their hydrolysis products and the metabolites produced upon fermentation, including 2′-FL, fucose, carbohydrates-2 (a longer DP GOS structure), lactose, galactose, glucose, fructose, acetic acid, lactic acid, formic acid, succinate, fumarate, ethanol, 1,2 propanediol, acetoin, indole-3-lactic acid.

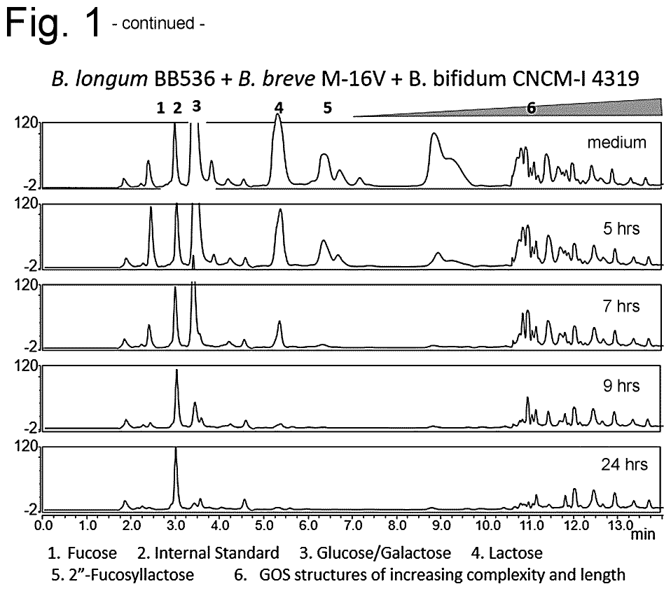

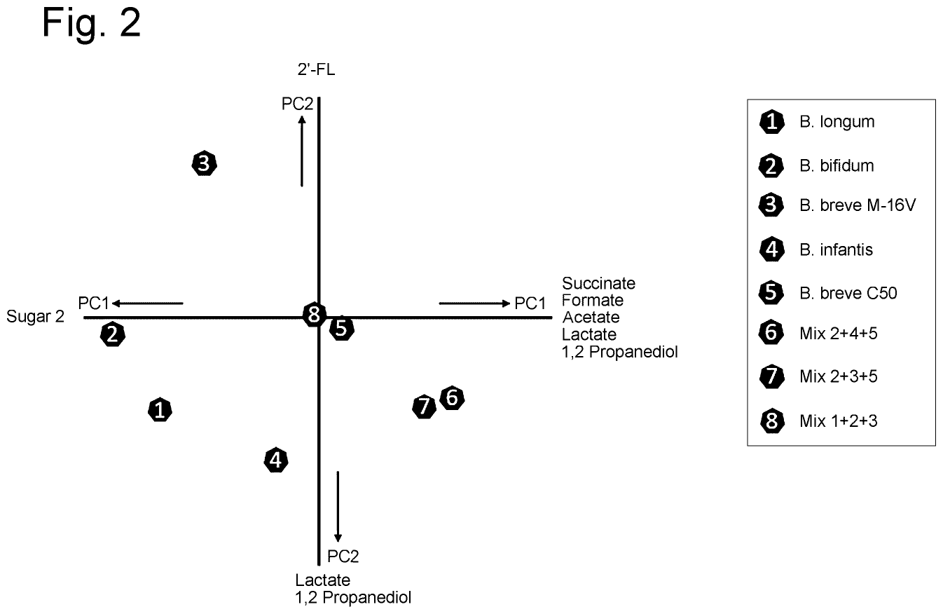

Results are shown in FIG. 2. Using Principal Component Analysis of the supernatants of the individual strains and the three mixes grown on GOS/FOS/2′-FL it was seen that Principle Component 1 to the right was significantly driven by formation of lactate, acetate, formate, 1,2 propanediol and succinate, indicating more efficient GOS/FOS/2′-FL fermentation. Lactate and acetate are the main metabolites from central carbohydrate metabolism of bifidobacterial. 1,2 Propenediol, formate and acetoin are linked to fucose metabolism. To the left of PC1 it was seen that there was no consumption of sugar-2 (a longer DP GOS structure). PC2 to the top was driven by 2′-FL, indicative of poor consumption of 2′-FL. PC2 to the bottom was more directed towards lactate and 1,2-propanediol (end product of fucose metabolism). The mixes of B. bifidum+B. breve C50+B. breve M-16V and B. bifidum+B. breve C50+B. infantis were much more directed in PC1 to the right and in PC2 to the bottom, indicating a much more efficient fermentation due to their synergistic interactions on GOS/FOS/2′-FL. The mix containing B. bifidum+B. longum and B. breve M-16V ended up in the middle of the PCA-plot being not able to ferment the long DP structures of GOS and further not able to (in case of B. longum) or not being as efficient (in case of B. breve M-16V) in fucose metabolism as B. breve C50. All the individual strains were much more directed to the left in PC1, indicating an incomplete or slower fermentation of the substrate was seen.

The patterns could be established, and relating to the amount of organic acids formed the results were again much in accordance with the NaOH consumption data and with the highest amount of acids formed with the mixes of the invention. The main organic acid formed was acetic acid, but also lactic acid was present in substantial amounts.

Related to the amounts measured by 1H-NMR the levels of 2′-FL, sugar-2 (a longer DP GOS structure), galactose, glucose and fucose the results were again very much in accordance with the glycoprofiling. These metabolites were indicative of a poor lactose or poor fucose metabolism. Only with B. bifidum as single strain fucose was accumulated in the supernatant. In test situations with B. breve C50 the higher DP bGOS was consumed. Interestingly, B. bifidum as single strain did not produce 1,2 propanediol, but in combination with B. breve 1,2 propanediol was formed, for all the three mixes tested (B. breve C50+B. bifidum+B. breve M-16V, B. breve C50+B. bifidum+B. infantis, B. breve M-16V+B. bifidum+B. longum) indicating that during the fucose was more efficiently catabolized into 1,2 propanediol, due to syntrophic interactions. Acetoin was observed in low amounts with the single strain of B. bifidum, but were higher with the other conditions. Interestingly indole-3-lactic acid was formed at the end of the fermentation stage. Indole-3-lactic acid is a metabolite associated with Bifidobacterium-dominated microbiota and linked with an anti-inflammatory effect on epithelial cells and considered an important mediator of host-microbial interactions. The highest levels were observed with the 3 mixes tested, when compared to the single strains.

### Example 4: Bifidobacterium Growth Stimulation Assays

### Material and Methods

Samples of the culture supernatant were taken during the fermentation experiments as described in examples 1 and 2. Samples were centrifuged for 10 min at 3250 g after which the supernatants were stored at −20° C. until analysis. Samples were centrifuged for 20 min at 13.000 g at RT. After that, supernatants were filtered using a low-binding filter column (0.22 μm, MILLEX GV Durapore PVDF membrane, Merck, Darmstadt, Germany) and the samples were stored at −20° C. until analysis.

The effect of the 24-hours fermentation supernatants (pH 6.5) was tested for growth stimulation of single Bifidobacterium strains. All supernatants were tested in flat bottom 96-wells plates (Falcon@, Corning, Tewksbury, USA) according to a template, which included positive, negative, internal and blanc controls. Each test well contained 20 vol % supernatant and 5 vol % of overnight Bifidobacterium culture in Colonic growth medium with 1 wt/v % lactose as its C-source.

Preculturing of bifidobacteria was performed in the same medium. The strains tested for growth stimulation were B. bifidum CNCM I-4319, B. breve M-16V, B. breve C50, B. longum BB536, and B. infantis BB-02.

The 96-wells plates were incubated anaerobically at 37° C. in the microplate reader (BioTek Powerwave HT, Biotek Instruments Inc., Winooski, USA) which performed kinetic measurements of OD600 every ten minutes for 24 hours. Growth curves were obtained and different parameters like maximum growth rate (μmax), time until μmax and the lag time were calculated using Gen5 software (Gen5 2.018, Biotek Instruments Inc., Winooski, USA). After checking and correcting these parameters for any possible outliers, the data were exported to Excel.

### Results

In Table 6 the effectiveness of bifidobacterial supernatants grown on GOS/FOS/2′-FL on bifidobacterial growth of individual Bifidobacterium strains is shown. Growth stimulation was expressed as ratio based on the growth stimulation compared to growth without supernatant. Growth curves have been obtained and three different parameters that represented influenced growth namely μmax, max OD and time until 50% OD was reached have been calculated as follows:

((μmax [strain control]/μmax [strain supernatant 1:5])+(Max OD [strain control]/Max OD [strain supernatant 1:5])+(1/(time until 50% OD [strain control]/time till 50% OD [strain supernatant 1:5]))) divided by 3.

For example, effect of supernatant from the mix of B. bifidum+B. breve 50+B. breve M-16V on the growth B. breve M-16V on GOS/FOS/2′-FL:

Ratio μmax is 1.60, ratio max OD is 0.97, ratio 1/(time until 50% OD was reached) 1.48, respectively, so the growth stimulation factor is 1.35.

TABLE 6
Growth stimulation of specific Bifidobacterium species by supernatant taken at t = 24 h from
cultures grown at pH 6.5 on culture medium containing GOS/FOS/2′-FL as the only carbohydrate source.

| | Supernatant of: | | | | | | |
|---|---|---|---|---|---|---|---|
| | | | | | | | B. breve |
| | | | | | B. breve | B. breve | C50 + |
| | | | | | M-16V + | C50 + | B. breve |
| | | B. breve | B. breve | | B. bifidum + | B. bifidum+ | M16V + |
| Strain tested | B. bifidum | C50 | M 16-V | B. infantis | B. longum | B. infantis | B. bifidum |
| B. bifidum | >2 | >2 | >2 | >2 | >2 | >2 | >2 |
| B. breve C50 | >2 | >2 | 1.5-2.0 | >2 | >2 | >2 | >2 |
| B. breve M-16V | 1.25-1.5 | 1.25-1.5 | 1.25-1.5 | 1.0-1.25 | 1.0-1.25 | 1.0-1.25 | 1.25-1.5 |
| B. infantis | 1.25-1.5 | 1.25-1.5 | 1.25-1.5 | 0.5-0.75 | 1.5-2.0 | 1.25-1.5 | 1.5-2.0 |
| B. longum | 1.0-1.25 | 1.0-1.25 | 1.0-1.25 | 0.5-0.75 | 1.0-1.25 | 0.75-1.0 | 1.25-1.5 |

The supernatants of the single strains stimulated growth of the bifidobacterial strains, except for the supernatant of the B. infantis strain. In that case an inhibitory effect on B. infantis and B. longum was observed. The supernatants of the single B. breve strains and the B. bifidum strain were growth stimulatory, especially for the growth of the B. bifidum strain.

Yet, the supernatant of the mix of these 3 strains was more effective in co-stimulating a broad spectrum of the single strains. Inhibition of B. infantis or B. longum was no longer observed. The best supernatant was from the mix of B. bifidum+B. breve C50+B. breve M-16V. This supernatant of the mix was especially effective in stimulating the growth of the B. infantis strain and B. longum strain when compared to the supernatant of the single strains and it improved the growth of B. longum to the highest extent compared to the other supernatants. Thereby being able to stimulate all four infant type bifidobacteria to the best extent.

In conclusion the supernatant of the specific mix, containing the metabolites as demonstrated in example, showed the best stimulation of the growth of the bifidobacteria, not only of the strains of the specific mix, but also of other Bifidobacterium species.

### Example 5: Pathogen Growth Inhibition

### Pathogen Growth Inhibition Assays

The influence of fermentation supernatants from individual Bifidobacterium strains and their mixes on pathogen growth has been tested in a pathogen inhibition assay. The supernatant of t=24 h was taken from example 2, growing on Colonic growth medium pH 5.5 and GOS/FOS/2′-FL as carbon and energy source. Growth curves of pathogenic bacteria could not been obtained by the same calculation method as used for the Bifidobacterium stimulation assay since with many pathogens not 50% of OD could be reached. Therefor it was decided to use the ratio of μmax.

A similar set up as for the bifidobacteria growth stimulation assay of example 4 was used, except that 1 wt/v % glucose was used as carbon- and energy source for the pathogens. The effect of the 24-hours fermentation supernatants of pH 5.5 were tested against several for infants relevant aerobic and anaerobic opportunistic pathogens. The results are shown in Table 7.

The supernatants of Bifidobacteria grown at pH 5.5 showed a strong inhibition on the growth of most of the tested pathogens. This pH is representative for the intestine of a breastfed infant or infant fed a formula containing a high amount of non-digestible oligosaccharides. The mix of B. breve C50, B. breve M-16V and B. bifidum CNCM I-4319 showed a somewhat stronger inhibitory effect on the growth of pathogens, in particular for the Gram-negative bacteria Y. enterocolitica, P. mirabilis, S. flexneri, and the Gram-positive bacteria L. monocytogenes, S. aureus. When looking at the average growth inhibition factor this for the 14 pathogenic strains was higher with the supernatant of the mix than with the supernatant of the individual strains.

TABLE 7
Inhibition of pathogenic bacteria by fermentation supernatants expressed
as growth ratio. The μmax ratio is the μmax observed with the
supernatant divided by the μmax in the control without supernatant.

| | | | | B. bifidum, |
|---|---|---|---|---|
| | pH 5.5 | B. breve | B. breve | B. breve |
| | B. bifidum | C50 | M16-V | C50 and M-16V |
| Y enterocolitisca | 0.05 | 0.20 | 0.14 | 0.02 |
| ATCC9610 | | | | |
| E coli EPEC | 0.16 | 0.17 | 0.20 | 0.26 |
| DSM9028 | | | | |
| P mirabilis | 0.21 | 0.08 | 0.12 | 0.06 |
| (clinical isolate) | | | | |
| S flexneri ATCC | 0.32 | 0.13 | 0.24 | 0.02 |
| 12022 | | | | |
| E coli EIEC ATCC | 0.28 | 0.26 | 0.43 | 0.30 |
| 35401 | | | | |
| S typhimurium | 0.21 | 0.12 | 0.15 | 0.22 |
| ATCC 14028 | | | | |
| E coli ETEC DSM | 0.19 | 0.32 | 0.30 | 0.30 |
| 8696 | | | | |
| C perfringens | 0.92 | 0.35 | 0.39 | 0.33 |
| ATCC 13124 | | | | |
| E faecalis ATCC | 0.38 | 0.45 | 0.50 | 0.40 |
| 4200 | | | | |
| L monocytogenes | 0.75 | 0.55 | 0.58 | 0.32 |
| ATCC 7644 | | | | |
| C sakazakii LMG | 0.60 | 0.50 | 0.61 | 0.52 |
| 2786 | | | | |
| S aureus ATCC | 0.98 | 0.64 | 0.77 | 0.45 |
| 29213 | | | | |
| K pneumoniae | 0.93 | 0.69 | 0.83 | 0.73 |
| clinical isolate | | | | |
| Average inhibition | 0.46 | 0.34 | 0.40 | 0.30 |

### Example 6: Fermentation of 2′-FL or GOS/FOS/2′-FL by a Faecal Sample of Antibiotic Treated Infant, Inoculated with Single Bifidobacterium Strains or a Mix

### Material and Methods

A faecal slurry fermentation experiment was performed wherein the behavior of different antibiotic resistance labelled bifidobacterial strains alone or in mixes in a microfluidics multifermentor system (BioLector Pro, M2P-Labs Germany) on different carbohydrates, was compared.

Tested were the singles strains, mixes and the carbon sources (2′-FL alone, GOS/FOS/2′-FL) as described in example 1 and 2. In comparison to example 1 and 2 not B. infantis BB-02 but B. infantis M63 was used, because of the natural resistance to streptomycin.

A faecal sample from a 5 month old breastfed infant was selected. This infant had been treated with antibiotics 2-3 weeks prior to faecal sampling. Under anaerobic conditions the faecal sample was thawed and a 4% (w/v) suspension of this faecal sample was made in adapted Colonic growth medium without carbon source was made. This Colonic growth medium at start contained an additional 25 mM acetate and 12 mM lactate, 25 mg/L bile acids (Sigma), 2.5 g/L porcine stomach mucin, 15 mmol/L ammonium sulphate in order to mimic infants' intestinal conditions and instead of 10 g, 1 g/I tryptone was present. During refeeding/refreshing medium, the medium did not contain acetate and lactate anymore since no accumulation of acetate and lactate was desired. The diluted faecal sample was homogenized, allowed to sediment for 5 minutes, then filtered over a tea sieve to remove large particles and subsequently filtered over a Millex 100 μm vacuum filter.

A 32 well Biolector Pro plate (BOH3 round well, M2P-labs) with optodes adapted that can handle the low pH range (pH 4-6) was used. All fermentation wells of this plate were filled with 1.6 ml of the faecal solution, one row of the plate was filled with sterile 3M NaOH. Twenty microliters of respectively i) B. bifidum CNCM I-4319+pDM1-Spec (spectomycin), ii) B. breve M-16V pNZ44-strep, iii) B. breve C50 pAM5-Tet (from frozen pellets), iv) overnight culture B. infantis M63 (intrinsic streptomycin resistance) or mixes thereof (1:1 or 1:1:1) were added to the wells. After mixing, 40 μl were taken for selective spot plating (T-0 samples). Plates were sealed with ventilated silicone foil with slits. The plate was incubated allowing 1 hour pH stabilization (85% moisture, 37 degrees, 6-800 rpm, anaerobic). Four hundred microliters of 10% (w/v) sterile carbohydrate solutions, i.e. GOS/FOS/2′-FL and 2′-FL, were added, or as a control 400 ul sterile water was added (controls without added carbohydrates). The experiment was started with pH at setpoint 5.8 with continuous pH measurement. The pH was controlled between pH 5.4-5.8. After 6 hours the experiment was paused, from each well 1500 μl was sampled (aseptically via slits in silicone foil), 40 μl of sample was used for selective spot plating.—The remainder of the sample was centrifuged. Remainder of the sample was centrifuged anaerobically and Pellets were resuspended in 1200 μl adapted Reichardt medium+300 μl of carbohydrate solution (or water) and pipetted back in BOH3 plate.

### Results

Results are shown in Table 8.

TABLE 8
NaOH consumed (mmol/liter) at t = 16 in a faecal slurry
obtained from an antibiotic treated infant, inoculated with single
strains of Bifidobacterium or a mix of Bifidobacterium strains,
and using as carbon source 2′-FL or GOS/FOS/2′-FL. NaOH
consumption was corrected the blanc (no carbohydrates added)

| | 2′-FL | GOS/FOS/2′-FL |
|---|---|---|
| strain | NaOH consumed | NaOH consumed |
| B. breve M16V | 57.8 | 58.7 |
| B. breve C50 | 57.3 | 57.4 |
| B. bifidum | 64.7 | 66.0 |
| B. infantis M63 | 57.3 | 59.7 |
| Mix B breve M-16V, B. breve | 64.8 | 68.7 |
| C50, B. bifidum | | |
| B. infantis, B. breve C50 | 52.7 | — |

Under conditions were no extra carbon and energy source was added, little or no growth and very little NaOH consumption was observed (data now shown, ranging from 3 to 6).

When 2′-FL alone was used as carbon and energy source, the combination of B. infantis+B. breve C50 showed less NaOH consumption than the single strain(s), and this is indicative of the antagonism also being present under conditions mimicking the intestinal microbiota environment with 2′-FL as carbohydrate. Of the single strains, the B. bifidum strain showed the highest additional NaOH consumption. The mix of 3 strains B. breve C50, B. breve M-16V and B. bifidum showed higher NaOH consumption compared to the single strains in intestinal microbiota exposed to a disruptive antibiotic treatment two weeks prior fecal sampling. This is indicative an improved effect of the specific mix of the invention.

With GOS/FOS/2′-FL as carbon and energy source a slightly higher NaOH consumption was observed than with 2′-FL alone. Of the single strains, again the B. bifidum resulted in the highest NaOH consumption. The mix of B. breve C50, M-16V and B. bifidum showed higher NaOH consumption compared to the single strains when grown on GOS/FOS/2′-FL and the level of NaOH consumed was higher than can be expected based on the single strains alone. This is indicative an improved effect of the specific mix of the invention on acid production under conditions where it is part of an intestinal microbiota, in particular in a dysbiotic intestinal microbiota or an intestinal microbiota that was exposed to a disruptive event like antibiotic treatment.

Plating revealed that the total amount of bifdobacteria is high and comparable in the presence of 2′-FL or GOS/FOS/2′-FL. Without a carbon and energy source the amount of bifidobacteria decreased after 6 h. The addition of the bifidobacterial strains did not have a large effect on the total bifidobacterial population during the experiment when fed GOS/FOS or 2′-FL, but for both carbon and energy sources the added bacteria became part of the total bifidobacteria population, as single strains or when added as mix of strains, as assessed by the spotplating. The mix with B. breve C50, B. breve M-16V and B. bifidum showed the strongest colonizer of the 3 mixes both with 2′-FL and with GOS/FOS/2′-FL (data not shown).

### Example 7: Fermentation of GOS/FOS and a HMO Mix Comprising 2′-FL in the Presence or Absence of a Mixture of Bifidobacterium Species, by Microbiota from a Caesarean Section Delivered Infant or a Vaginally Born Infant or by Antibiotic Vs Non Antibiotic Treated Microbiota

### Faecal Slurry Fermentation

### Faecal Samples

Faecal samples from monozygotic twin, were freshly collected from both infants on the same day and immediately frozen at −80 degrees Celcius. The infants were exclusively breastfed, 2.5 months of age, one was born vaginally while the other was born by non-elective caesarean (C-)section.

Samples were at the start of the experiment defrosted under anaerobic conditions in anaerobic cabinet. Samples for preculturing the uncompromised (non-antibiotic treated) microbiota were approx. 1:100 diluted in Colonic growth medium (adjusted to uncompromised breastfed infant stool pH (pH 5.5)) containing, 25 mM acetate and 12 mM lactate, 25 mg/L bile acids (Sigma), 2.5 g/L porcine stomach mucin, 15 mmol/L ammonium sulphate, 1 g/I tryptone and 5 g/L lactose in order to mimic infants' uncompromised intestinal conditions. The same sample was used for preculturing the compromised (antibiotic treated) microbiota, again the sample was diluted 1:100 in Colonic growth medium adjusted to higher infant stool pH more often seen in infants of which the microbiota is adversely predisposed, namely pH 6.5, and positive factors naturally present in uncompromised microbiome selective components (acetate, lactate and bile salts) were not added to medium, while the carbon source was replaced by 5 g/L glucose. During pre-fermentation of the compromised microbiota the sample was treated with the antibiotic clindamycin in a concentration of 1 microgram/ml. Clindamycin suppresses largely the anaerobic gram-positive bacteria and is commonly used in infants when antibiotic treatment is necessary.

Briefly, B. bifidum CNCM-I 4319, B. infantis BB-02 and B. breve C50 were cultured in DasGib system in Basal growth medium (10 g/L tryptone, 1 g/L yeast extract, 3 g/L potassium dihydrogen phosphate, 4.8 g/L dipotassium hydrogen phosphate, 3 g/L Ammonium sulphate, 0.2 g/L Magnesium sulphate heptahydrate, 0.5 g/L L-Cysteine hydrochloride, monohydrate) supplemented with 5 g/L GOS/FOS/HMO, under anaerobic conditions. The pH was controlled at pH 6.5 with 5M NaOH. At end logarithmic phase bacterial cells were harvested and concentrated droplets were made in liquid nitrogen. The CFU counts were done. As source of carbohydrates a mixture of GOS/FOS/2′-FL was used in a wt/wt/wt ratio of about 7.5:1:1.5. The carbohydrate solution had a 10 wt % final concentration.

The microbiota of the pre-cultures were harvested by centrifuging and taken up in concentrated Colonic growth medium (pH5.6) without carbon source (similar as in uncompromised preculture), divided over 2 tubes. To one of the tubes the bifidobacterial strains were added in a 1:1:1 ratio based on cfu and a final concentration of 1×10⁸ CFU/ml.

The fermentations were performed in a microfluidics multifermentor system (BioLector Pro, M2P-Labs Germany).

A 32 well Biolector Pro plate (BOH2 round well, M2P-labs) with pH optodes was used. Half of the fermentation wells of this plate were filled with 1.6 ml of the faecal solution without Bifidobacterium mixture, while other half of the plate was filled with faecal solution with the Bifidobacterium mixture. One feeding row of the plate was filled with sterile 3M NaOH, the other feeding row was filled with either 10% carbohydrate (CHO) solution or blanc solution.

This feeding well was only used during the night feeding. pH setpoint during fermentation was pH 5.6-5.8. Plates were sealed with ventilated silicone foil with slits. First feeding of the faecal slurry (except for night feeding) was manually and in a controlled timely mode, mimicking more or less the intestinal feeding of the colon of the infant. Meaning at start 20 μl 10% CHO solution (25%), after 1 hour 40 μl 10% CHO solution (50%) and after 2 hours again 20 μl 10% CHO solution (25%). The total dose of one feeding is 5 g/L of carbohydrate (or the same volume for blanc). Such a feeding regime is thought to closely mimic the feeding dose of infants. At 3 hours the faecal supernatants of each fermenter well was collected by centrifuging for further analyses, while the microbiota (pellet) was used to start an identical 2^{nd} feeding procedure. In total there were 3 feeding sessions during 2 days, so 6 feeding sessions of 3 hr in total and during the night there was a continuous automatic linear feeding mode providing in total one dose (5 g/L) of the carbohydrates (GOS/FOS/2′-FL mix or same volume blanc). At regular intervals 40 μl of sample was used for selective Bifidobacterium spot plating. Bifidobacteria were determined as described above.

### Results

The results are given in table 9 for both infants of the twins, the vaginally born infant (infant A) and C-section born infant (infant B).

The total amount of NaOH consumed after all 7 feedings upon fermentation of the GOS/FOS/2-′FL in the absence of bifidobacteria was lower in the microbiota of baby B, the C-section born infant, and about 88% compared to the value observed for the microbiota of the vaginally born twin sibling, baby A. When a mixture of bifidobacteria was present, the total NaOH consumption increased in the microbiota of both infants. The increase was about 8% for microbiota of baby A and about 24% for the microbiota of baby B. Strikingly, in the presence of the bifidobacteria mix the NaOH consumption became similar (101%) in baby B vs baby A.

In antibiotic treated microbiota the total amount of NaOH consumed upon fermentation of the GOS/FOS/2′-FL in the absence of bifidobacteria was much lower (56%) when compared to total NaOH consumption of the non-antibiotic treated microbiota. When a mixture of bifidobacteria was added, the total NaOH consumption increased by about 43% compared to when such a mixture was not added. In the presence of added bifidobacteria the amount of NaOH consumed was about 64% compared with non-antibiotic treated microbiota, and this is indicative of a relative increased restoration of the microbiota function.

TABLE 9
NaOH consumed (mmol/l) during each feeding interval in non-antibiotic treated
faecal slurries obtained from twin infants of which one was born via C-section
delivery (baby B) and one was born vaginally (baby A), using as carbon source
GOS/FOS/2′-FL with or without a specific bifidobacterium mix

| | Non | Non | Non | Non | | |
|---|---|---|---|---|---|---|
| | antibiotic | antibiotic | antibiotic | antibiotic | Antibiotic | Antibiotic |
| | treated | treated | treated | treated | treated | treated |
| | microbiota | microbiota | microbiota | microbiota | microbiota | microbiota |
| | baby A | baby A | Baby B | Baby B | baby B | baby B |
| | GOS/FOS/2′- | GOS/FOS/2′- | GOS/FOS/2′- | GOS/FOS/2′- | GOS/FOS/2′- | GOS/FOS/2′- |
| | FL (5 g/L) | FL (5 g/L) | FL (5 g/L) | FL (5 g/L) | FL (5 g/L) | FL (5 g/L) |
| | bifididobact. | no added | bifididobact. | no added | bifididobact. | no added |
| Feeding | mix added | bifididobact. | mix added | bifididobact | mix added | bifididobact |
| 1 | 237.8 | 219.2 | 235.7 | 201.0 | 87.2 | 72.8 |
| 2 | 187.5 | 156.8 | 218.8 | 189.2 | 100.9 | 66.0 |
| 3 | 165.4 | 150.6 | 176.3 | 138.8 | 77.3 | 53.1 |
| 4 ON | 470.3 | 444.0 | 466.9 | 337.5 | 406.9 | 225.0 |
| 5 | 191.3 | 180.8 | 172.1 | 158.6 | 96.4 | 79.7 |
| 6 | 190.1 | 177.0 | 165.0 | 139.1 | 148.5 | 117.2 |
| 7 | 168.9 | 167.4 | 192.2 | 144.9 | 131.6 | 117.8 |
| sum | 1611.3 | 1495.8 | 1627.0 | 1309.1 | 1048.8 | 731.6 |

ON = overnight feeding

When no GOS/FOS/2′-FL mix was added, hardly any NaOH consumption was observed (data not shown).

For non-antibiotic microbiota the total number of bifidobacteria at t=0 was lower in the microbiota of baby B born via C-section than that of the microbiota of baby A who was vaginally born (log cfu/ml 8.19 vs 8.62). In the presence of GOS/FOS/2′-FL mix the amount of bifidobacteria slightly increased in time. The amount of bifidobacteria was also slightly higher under conditions when the bifidobacterial mix was added vs conditions where no such addition was made. The amount of bifidobacteria in time became similar for the microbiota of both baby A and B.

In antibiotic treated microbiota of baby B the amount of bifidobacteria was low (log cfu/ml 6.35 vs 8.19) and in the absence of added bifidobacteria the amount of bifidobacteria slightly increased. In the presence of added bifidobacteria the amount of bifidobacteria at t=0 was lower compared to non-antibiotic treated, (log cfu/ml 7.95 vs 8.43) but in time the number of bifidobacteria increased and became comparable to the number of bifidobacteria in the microbiota of non-antibiotic treated vaginally born baby A (log cfu·ml 8.77 vs 8.98).

In the absence of GOS/FOS/2′-FL the level of bifidobacteria slightly decreased.

Consumption of NaOH is indicative of the amount of acids (like short chain fatty acids and lactate) that is produced upon fermentation of the carbohydrates (GOS/FOS/2′-FL) by the microbiota. Therefore these results are indicative for a high acid formation, being slightly higher in the vaginally born infant than in the C-section born infant. Upon addition of the Bifidobacterium mix comprising B. bifidum able to express at least one extracellular enzyme selected from a fucosidase, and a sialidase and B. breve able to metabolize a saccharide selected from L-fucose and sialic acid, the acid formation is increased relatively to a higher degree in the microbiota of the C-section born infant. Likewise, similar positive effects on the amount of bifidobacterial in the microbiota were observed when comparing these groups. This is indicative of preventing and/or treating intestinal microbial dysbiosis in an infant born via caesarean section.

The effect of antibiotic treatment is indicative of a reduced acidification. This acidification was increased to a relatively higher extent when the mix of bifidobacteria of the invention was also present, compared to non-antibiotic treated microbiota. Likewise, similar positive effects on the amount of bifidobacterial in the microbiota were observed when comparing these groups. This is indicative for a positive effect on preventing and/or treating intestinal microbial dysbiosis in an infant exposed to antibiotics (either directly or via the mother) and also indicative for increasing the microbiota resilience against and recovery after exposure to a microbiota disturbing event, wherein the microbiota disturbing event is an antibiotic treatment.

### Example 8

Infant formula, in powder form, comprising per 100 kcal:
2.0 g protein (whey protein:casein 6:4)
5.0 g lipids (mainly vegetable lipids)
11.2 g digestible carbohydrates (mainly lactose)
non digestible oligosaccharides:
   1 g GOS (coming from Vivinal® GOS)
   0.12 g IcFOS (Raftiline® HP)
   0.24 g 2′-FL (Jennewein)
probiotics, being present at 10³-10⁹ cfu/g powder and comprising:
   B. breve C50
   B. breve M-16V
   B. bifidum CNCM I-4319
minerals, vitamins and other micronutrients as known in the art and according to guidelines for infant formulas.

The powder is in a pack that contains instructions to dilute with water and the ready to drink formula has 67 kcal/100 ml.

### Example 9

Follow on formula, in powder form, comprising per 100 kcal:
2.0 g protein (whey protein:casein 6:4)
4.7 g lipids (mainly vegetable lipids)
11.8 g digestible carbohydrates (mainly lactose)
non digestible oligosaccharides:
   1 g GOS (coming from Vivinal® GOS)
   0.11 g 2′-FL (Jennewein)
probiotics, being present at 10³-10⁹ cfu/g powder and comprising:
   B. breve MCC1274 (B-3 from Morinaga)
   B. bifidum R0071 (Rosell Lallemand)
minerals, vitamins and other micronutrients as known in the art and according to guidelines for infant formulas.

The powder is in a pack that contains instructions to dilute with water and the ready to drink formula has 67 kcal/100 ml.

### Example 10

Young child formula, in powder form, comprising per 100 kcal:
2.0 g protein (whey protein:casein 6:4)
4.0 g lipids (mainly vegetable lipids)
13.4 g digestible carbohydrates (mainly lactose)
non digestible oligosaccharides:
   1 g GOS (coming from Vivinal® GOS)
   0.11 g IcFOS (Raftiline® HP)
   0.11 g 3′-SL (GeneChem)
Probiotics, being present at 10³-10⁹ cfu/g powder and comprising:
   B. breve C50
   B. bifidum CNCM I-4319
Minerals, vitamins and other micronutrients as known in the art

The powder is in a pack that contains instructions to dilute with water and the ready to drink formula has 65 kcal/100 ml.

1-19. (canceled)

20. A nutritional composition comprising a mix of Bifidobacterium species and at least one human milk oligosaccharide, wherein
a. the Bifidobacterium species comprise at least i) a Bifidobacterium bifidum strain able to express at least one extracellular enzyme selected from a fucosidase, and a sialidase, ii) a Bifidobacterium breve strain able to metabolize a saccharide selected from L-fucose and sialic acid, and
b. the human milk oligosaccharide is at least one selected from the group consisting of 2′-fucosyllactose, 3-fucosyllactose, 3′-sialyllactose, and 6′-sialyllactose, and
wherein the nutritional composition further comprises galacto-oligosaccharide comprising beta1,4linkages having a degree of polymerization (DP) of at least 4, and wherein the nutritional composition is not mammalian milk.

21. The nutritional composition according to claim 20, comprising at least 0.005 g/100 ml of the human milk oligosaccharide.

22. The nutritional composition according to claim 20, wherein the human milk oligosaccharide is 2′-fucosyllactose and wherein the Bifidobacterium breve strain is able to metabolize L-fucose.

23. The nutritional composition according claim 20, comprising at least 0.01 g/100 ml 2′-fucosyllactose.

24. The nutritional composition according claim 20, wherein the Bifidobacterium breve strain is able to express extracellular beta1,4endogalactanase.

25. The nutritional composition according to claim 20, wherein the amount of galacto-oligosaccharide comprising beta1,4linkages having a DP of at least 4 is at least 50 mg/100 ml.

26. The nutritional composition according to claim 20, further comprising non-digestible polyfructose.

27. The nutritional composition to claim 20, comprising a strain of B. breve that is able to express extracellular beta1,4endogalactanase and a strain that is not able to express beta1,4endogalactanase.

28. The nutritional composition according claim 20, wherein the Bifidobacterium breve strain or strains do not express fucosidase and do not express sialidase.

29. The nutritional composition according to claim 20, further comprising a strain of Bifidobacterium longum subspecies infantis.

30. The nutritional composition according to claim 20, wherein the nutritional composition does not comprise a Lactobacillus species.

31. The nutritional composition according to claim 20, that contains at least 10⁵ cfu B. bifidum per gram dry weight of the nutritional composition and at least 10⁵ cfu B. breve able to express beta1,4endogalactanase, per gram dry weight of the nutritional composition and at least 10⁵ cfu B. breve not able to express beta1,4endogalactanase per gram dry weight of the nutritional composition, and optionally contains B. infantis, and wherein the total amount of bifidobacteria is at least 10⁶ cfu per gram dry weight of the nutritional composition.

32. The nutritional composition according to claim 20, wherein the nutritional composition further comprises protein, carbohydrates, lipids, vitamins and minerals and is a liquid or a powder suitable to be reconstituted to a liquid, and the nutritional composition is preferably an infant formula, a follow on formula or a young child formula.

33. A method of preventing and/or treating intestinal microbial dysbiosis and/or reducing intestinal pathogenic bacteria in a subject, preferably a child, preferably in an infant, the method comprising administering the nutritional composition to the child.

34. The method according to claim 33, wherein the subject is selected from the group consisting of a subject that was born preterm, a subject that was born via caesarean section, a subject that is or has been treated with antibiotics and a subject that is or has been at least partly breastfed by a woman taking antibiotics, wherein the human subject is a child, preferably in an infant.

35. A method for reducing the risk of occurrence, preventing and/or treating an intestinal infection, intestinal inflammation and/or diarrhea in a child, preferably in an infant, the method comprising administering the nutritional composition to the child.
